(19) United States
(12) Patent Application Publication
(10) Pub. No.: US 2005/0014996 A1
(43) Pub. Date: Jan. 20, 2005

(54) Optical adaptor and endoscope device

(76) Inventors: Yutaka Konomura, Tokyo (JP); Kiyotomi Ogawa, Tokyo (JP)

Correspondence Address: OSTROLENK FABER GERB & SOFFEN, 1180 AVENUE OF THE AMERICAS, NEW YORK, NY, 100368403, US

(21) Appl. No.: 10/820,203

(22) Filed: Apr. 7, 2004

(30) Foreign Application Priority Data
Apr. 11, 2003 (JP) P2003-107674

(51) Int. Cl. A61B 1/04

(52) U.S. Cl. 600/175; 600/118

(57) ABSTRACT

An optical adaptor and an endoscope device provides a means which can identify an optical adaptor to be used accurately, thus enabling misoperation by a user to be prevented. A construction is adopted wherein an optical adaptor incorporates an IC chip in which identification information and optical characteristic information of the optical adaptor is stored, and the tip of an endoscope insertion section is provided with an antenna which obtains the identification information and optical characteristic information from the identification IC chip.

### BACKGROUND OF THE INVENTION

1. Field of the Invention

The present invention relates to an optical adaptor to be installed at the tip section of the insertion section of an endoscope, and an endoscope device that is provided with this optical adaptor.

Priority is claimed on Japanese Patent Application No. 2003-107674, filed on Apr. 11, 2003, the content of which is incorporated herein by reference.

2. Description of Related Art

Industrial endoscope devices are used for a range of purposes such as the inspection of aircraft engine blades, internal inspection of electric power conduits, and the like. The characteristics of this industrial endoscope device are that the endoscope insertion section, which has an imaging section at its tip, is longer than that of medical ones, and an optical adaptor mounted on the imaging section can be changed according to the inspection purpose.

This type of endoscope device comprises, basically, an endoscope to be inserted in an object to be examined, a light source for supplying illuminating light to a light guide incorporated in this endoscope, a control unit for generating an image signal based on an electrical signal from a CCD (charge coupled device) incorporated in the tip of the endoscope, a television monitor for displaying the image signal, and the like, as shown in Japanese Unexamined Patent Application, First Publication No. H08-201706 (FIG. 1, FIG. 2, etc.)

An optical adaptor having an optical system which forms an image on a CCD is detachably installed at the tip of the endoscope. A plurality of types of optical adaptor exists depending on the observation purpose, such as stereoscopic observation, enlarged/wide-angle observation, and the like, and a user can choose the most suit able one according to his observation purpose.

In the case where an object to be examined is measured using such an endoscope device, when the control unit converts an electrical signal from the CCD to an image signal, it is necessary to understand the type and optical characteristics of the mounted optical adaptor in advance. The optical characteristics of the optical adaptor comprise a range of correction coefficients obtained in a situation where it is mounted on an endoscope device, which serves as a master, at the time of manufacture, as well as installation position information at that time, and the like. The optical characteristics of these optical adaptors are administered based on identifiers assigned to the optical adaptors.

Accordingly, when selecting an optical adaptor, the user selects a corresponding optical characteristic to be read by the control unit by inputting the identifier assigned to the optical adaptor, to the endoscope device. It is then possible to perform highly accurate measurements.

### SUMMARY OF THE INVENTION

An optical adaptor of the present invention is an optical adaptor that is detachably installed at the tip of an insertion section of an endoscope having a light receiving section at the tip thereof. The optical adapter includes: an optical system which forms an image in the light receiving section; and an information device containing at least one of information for identifying itself and optical characteristic information.

An endoscope device of the present invention includes: an endoscope insertion section having a light receiving section at the tip thereof; an optical adaptor that is detachably installed at the tip of the insertion section, and having an optical system which forms an image in the light receiving section, and an information device containing at least one of information for identifying itself and optical characteristic information; and a reading section which is installed at the tip of the endoscope insertion section and obtains this information.

An endoscope device of the present invention includes: a main body; an endoscope insertion section, which is connected to the main body and has a light receiving section at the tip thereof; an optical adaptor that is detachably installed at the tip of the insertion section, and having an optical system which forms an image in the light receiving section, and an information device containing at least one of information for identifying itself and optical characteristic information; and a reading section which is installed in the main body and obtains the information from the optical adaptor.

### BRIEF DESCRIPTION OF THE DRAWINGS

FIG. 1 is a diagram showing a first embodiment of an endoscope device of the present invention, being a perspective view showing its overall structure.

FIG. 2 is a block diagram showing the internal structure of the endoscope device.

FIG. 3 is a cross-sectional diagram showing a tip section of an endoscope insertion section provided in the endoscope device, and an optical adaptor installed therein.

FIG. 4 is a cross-sectional diagram showing another cross section of the tip section of the endoscope insertion section and the optical adaptor.

FIG. 5 is a cross-sectional diagram through A-A of FIG. 4 showing a joining surface between the tip section of the endoscope insertion section and the optical adaptor.

FIG. 6 is a block diagram of an electrical circuit provided in the endoscope device.

FIG. 7 is an explanatory diagram to explain the exchange of communication data between a CPU and identification IC chips provided in the endoscope device.

FIG. 8 is a diagram showing the format of communication data exchanged between the CPU and an identification IC chip.

FIG. 9 is a diagram showing a second embodiment of the endoscope device of the present invention, being a cross-sectional diagram showing the tip section of the endoscope insertion section, and an optical adaptor installed therein.

FIG. 10 is a block diagram of an electrical circuit provided in the endoscope device.

FIG. 11 is a diagram showing a third embodiment of the endoscope device of the present invention, being a cross-sectional diagram showing the tip section of the endoscope insertion section, and an optical adaptor installed therein.

FIG. 12 is a block diagram of an electrical circuit provided in the endoscope device.

FIG. 13 is a graph showing the voltage of the electrical circuit of the endoscope device, wherein the horizontal axis shows frequency, and the vertical axis shows voltage.

FIG. 14 is a diagram showing a fourth embodiment of the endoscope device of the present invention, being a cross-sectional diagram showing the tip section of the endoscope insertion section, and an optical adaptor installed therein.

FIG. 15 is a block diagram of an electrical circuit provided in the endoscope device.

FIG. 16 is a diagram showing a fifth embodiment of the endoscope device of the present invention, being a cross-sectional diagram showing the tip section of the endoscope insertion section, and an optical adaptor installed therein.

FIG. 17 is a block diagram of an electrical circuit provided in the endoscope device.

FIG. 18 is a diagram showing a sixth embodiment of the endoscope device of the present invention, being a cross-sectional diagram showing the tip section of the endoscope insertion section, and an optical adaptor installed therein.

FIG. 19 is a block diagram of an electrical circuit provided in the endoscope device.

FIG. 20 is a diagram showing a seventh embodiment of the endoscope device of the present invention, being a cross-sectional diagram showing the tip section of the endoscope insertion section, and an optical adaptor installed therein.

FIG. 21 is a block diagram of an electrical circuit provided in the endoscope device.

FIG. 22 is a diagram showing an eighth embodiment of the endoscope device of the present invention, being a block diagram showing the internal structure.

FIG. 23 is a perspective view showing the location of an identification section provided in the endoscope device.

FIG. 24 is a cross-sectional diagram showing the identification section of the endoscope device.

FIG. 25 is a block diagram of an electrical circuit provided in the endoscope device.

FIG. 26 is a cross-sectional diagram showing the tip section of the endoscope insertion section provided in the endoscope device, and an optical adaptor installed therein.

FIG. 27 is a diagram showing a ninth embodiment of the endoscope device of the present invention, being a cross-sectional diagram showing the tip section of the endoscope insertion section, and an optical adaptor installed therein.

FIG. 28 is a cross-sectional diagram showing a situation in which the optical adaptor of the endoscope device is inserted in the identification section.

FIG. 29 is a block diagram of an electrical circuit provided in the endoscope device.

FIG. 30 is a diagram showing a tenth embodiment of the endoscope device of the present invention, being a cross-sectional diagram showing the tip section of the endoscope insertion section, and an optical adaptor installed therein.

FIG. 31 is a cross-sectional diagram showing a situation in which the optical adaptor of the endoscope device is inserted in the identification section.

FIG. 32 is a block diagram of an electrical circuit provided in the endoscope device.

FIG. 33 is a graph showing the voltage of the electrical circuit of the endoscope device, wherein the frequency is on the horizontal axis, and the voltage is on the vertical axis.

FIG. 34 is a diagram showing an eleventh embodiment of the endoscope device of the present invention, being a cross-sectional diagram showing the tip section of the endoscope insertion section, and an optical adaptor installed therein.

FIG. 35 is a cross-sectional diagram showing a situation in which the optical adaptor of the endoscope device is inserted in the identification section.

FIG. 36 is a block diagram of an electrical circuit provided in the endoscope device.

FIG. 37 is a diagram showing a twelfth embodiment of the endoscope device of the present invention, being a cross-sectional diagram showing the tip section of the endoscope insertion section, and an optical adaptor installed therein.

FIG. 38 is a cross-sectional diagram showing a situation in which the optical adaptor of the endoscope device is inserted in the identification section.

FIG. 39 is a block diagram of an electrical circuit provided in the endoscope device.

FIG. 40 is a diagram showing a thirteenth embodiment of the endoscope device of the present invention, being a cross-sectional diagram showing the tip section of the endoscope insertion section, and an optical adaptor installed therein.

FIG. 41 is a cross-sectional diagram showing a situation in which the optical adaptor of the endoscope device is inserted in the identification section.

FIG. 42 is a block diagram of an electrical circuit provided in the endoscope device.

FIG. 43 is a diagram showing a fourteenth embodiment of the endoscope device of the present invention, being a cross-sectional diagram showing the tip section of the endoscope insertion section, and an optical adaptor installed therein.

FIG. 44 is a cross-sectional diagram showing a situation in which the optical adaptor of the endoscope device is inserted in the identification section.

FIG. 45 is a block diagram of an electric al circuit provided in the endoscope device.

FIG. 46 shows pixel arrangement diagrams of an image before and an image after the correction of geometric distortion in the endoscope device of the present invention.

FIG. 47 is a diagram showing image pixels before correction and image pixels after correction in the endoscope device of the present invention.

### DETAILED DESCRIPTION OF THE INVENTION

Hereunder are descriptions of embodiments of an optical adaptor and an endoscope device of the present invention with reference to the figures. However, needless to say the present invention is not limited to these embodiments. Any structural addition, omission, replacement, or other modification that does not depart from the gist of the present invention is possible. The present invention is not limited by the following descriptions, and is limited only by the scope of appended claims.

(First Embodiment)

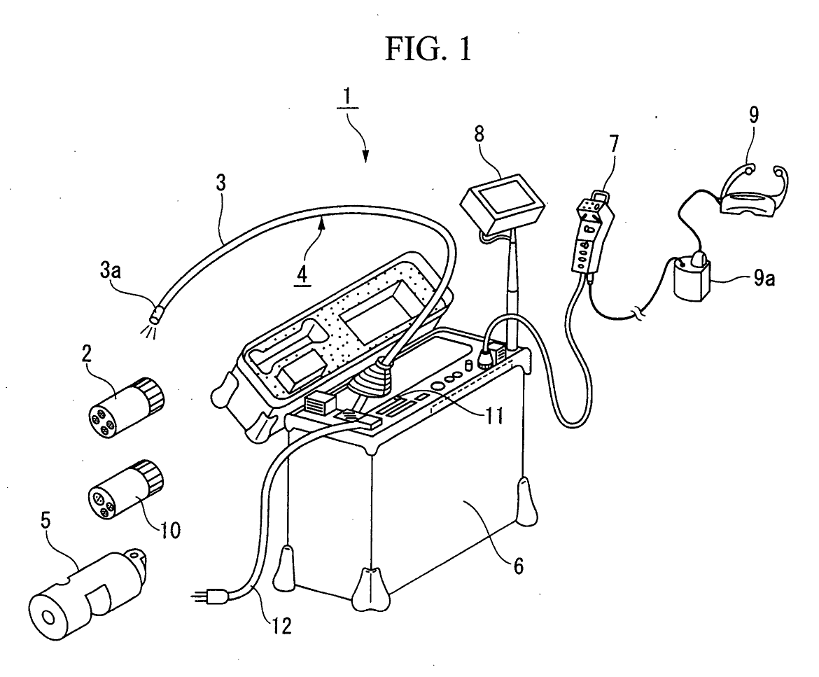

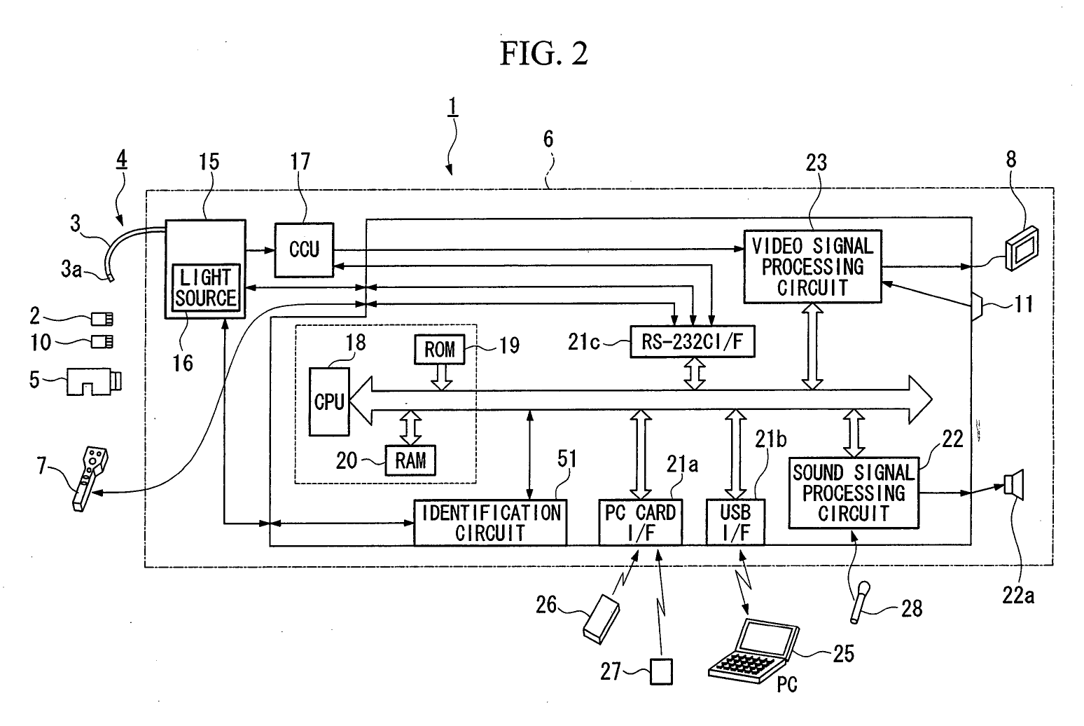

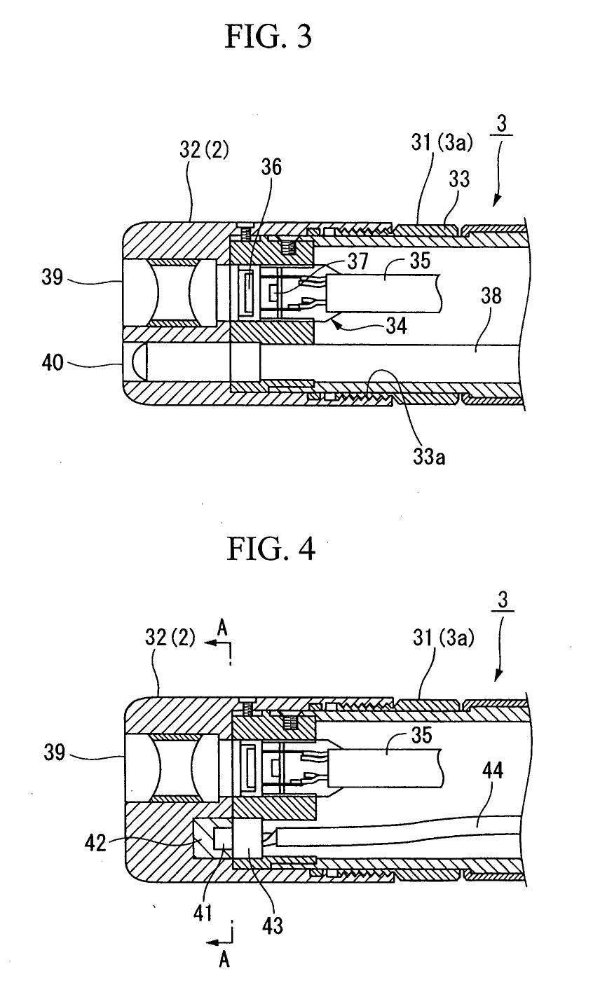

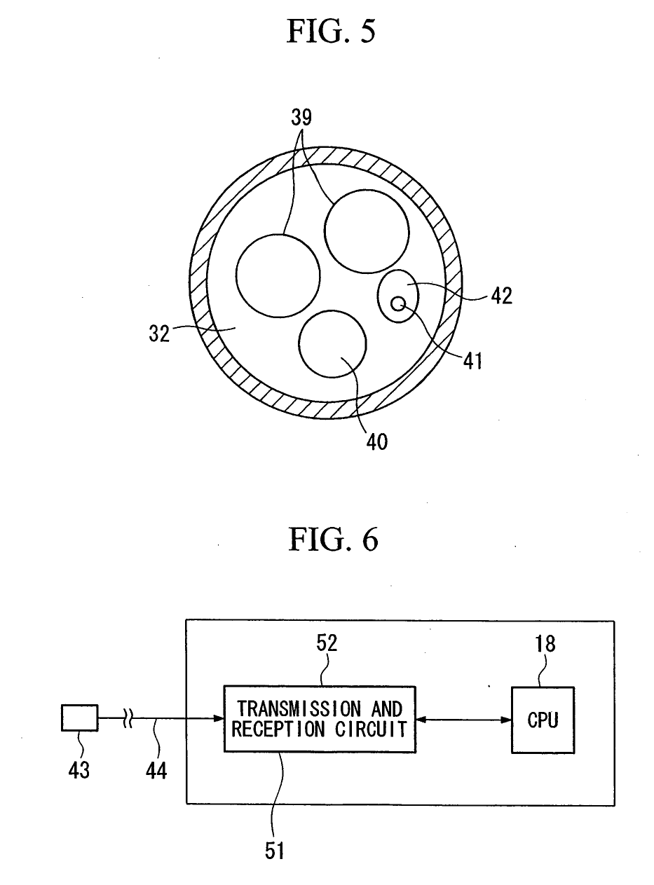

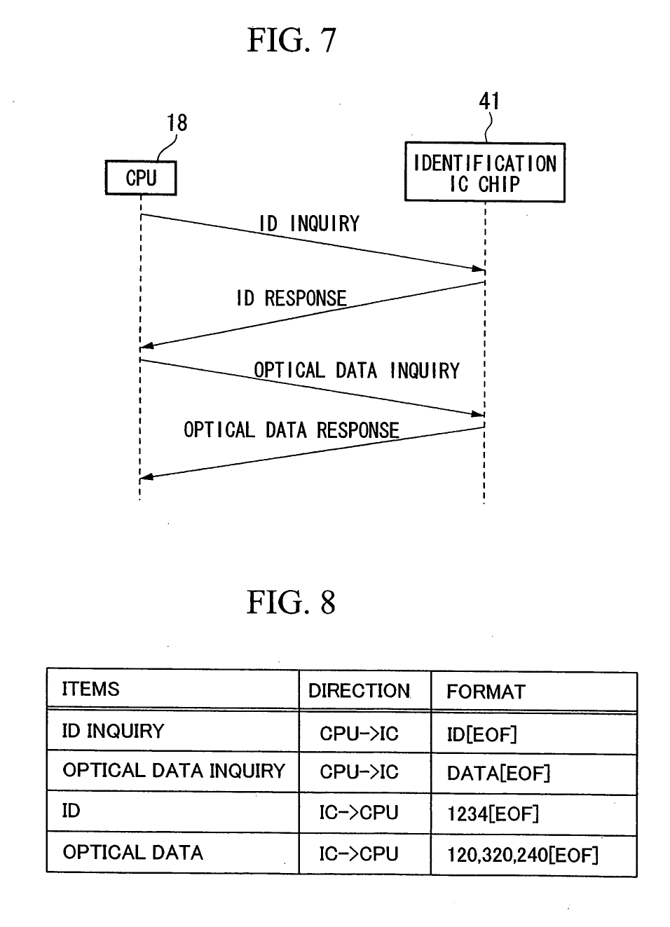

Hereunder is a description of a first embodiment of the present invention with reference to FIG. 1 to FIG. 8. FIG. 1 is a perspective view showing the overall structure of an endoscope device of the present embodiment. FIG. 2 is a block diagram showing the internal structure of the endoscope device. FIG. 3 is a cross-sectional diagram showing a tip section of an endoscope insertion section provided in the endoscope device, and an optical adaptor installed therein. FIG. 4 is a cross-sectional diagram showing another cross section of the tip section of the endoscope insertion section and the optical adaptor. FIG. 5 is a cross-sectional diagram through A-A of FIG. 4 showing a joining surface between the tip section of the endoscope insertion section and the optical adaptor. FIG. 6 is a block diagram of an electrical circuit provided in the endoscope device. FIG. 7 is an explanatory diagram to explain the exchange of communication data between a CPU and transmission and reception circuits provided in the endoscope device. FIG. 8 is a diagram showing the format of communication data exchanged between the CPU and the transmission and reception circuits.

Firstly, the system structure of an endoscope device 1 of the present invention will be described with reference to FIG. 1.

As shown in FIG. 1, this endoscope device 1 basically comprises a stereo measurement optical adaptor (optical adaptor) 2, an endoscope 4 having an endoscope insertion section 3 to which this stereo measurement optical adaptor 2 is detachably connected, a calibration tool 5 for capturing the shape of a mask in the stereo measurement optical adaptor 2, a control unit (main body) 6 in which the endoscope 4 is stored, a remote controller 7 for performing a range of motion control, a liquid crystal monitor (referred to hereunder as LCD) 8, being a display for displaying the endoscope image, operation control content (for example, processing menus), and the like, a face mount display (referred to hereunder as FMD) 9 capable of viewing a normal endoscope image stereoscopically, or a stereo image of the endoscope image, and a FMD adaptor 9a for supplying image data to this FMD 9.

The endoscope insertion section 3 is a narrow cable incorporating an imager (described later) at its tip section 3a, and can be inserted in a section to be examined. As well as the stereo measurement optical adaptor 2, a comparative measurement optical adaptor 10 may be detachably connected to the tip section 3a of the endoscope insertion section 3.

The calibration tool 5 is a tool into which the tip section 3a of the endoscope insertion section 3, on which the stereo measurement optical adaptor 2 is mounted, is inserted to capture the shape of a mask in the stereo measurement optical adaptor 2.

Reference numeral 11 in the figure denotes an external image input terminal for inputting an image to a video signal processing circuit without passing through a CCU 17 described later. Furthermore, numeral 12 denotes a mains cable for connecting to external electric power.

Next is a detailed description of the internal structure of the endoscope device 1 with reference to FIG. 2.

As shown in the figure, the proximal end section of the endoscope insertion section 3 is connected to an endoscope unit 15 in the control unit 6. This endoscope unit 15 incorporates a light source 16 for supplying the light required at the time of image pickup, and an electric bending apparatus (not shown in the figure) for electrically bending a bending section (not shown in the figure) incorporated in the endoscope insertion section 3.

Furthermore, a CCD (imager) 36 as described later is incorporated in the tip section 3a of the endoscope insertion section 3. An image signal output from this CCD 36 is input to a camera control unit (referred to hereunder as CCU) 17 being an image processing section. The construction of this CCU 17 is such that an input image signal is converted for example to a video signal, such as an NTSC signal or the like, and supplied to main processing circuits in the control unit 6.

The main processing circuits incorporated in the control unit 6 comprise; a CPU 18, a ROM 19, a RAM 20, a PC card interface (referred to hereunder as PC card I/F) 21a, a USB interface (referred to hereunder as USB I/F) 21b, an RS-232C interface (referred to hereunder as RS-232C I/F) 21c, a sound signal processing circuit 22, a video signal processing circuit 23, and an identification circuit 51.

The CPU 18 is a microprocessor serving as a control section for executing and operating a range of functions based on a main program, and an arithmetic processing unit for performing measuring processes. Moreover the CPU 18 performs motion control of the overall system by executing programs stored in the ROM 19, and performing processing as required for that purpose.

The RS-232C I/F 21c is an interface for performing the communication required for motion control of the CCU 17 and the endoscope unit 15 based on operation by the remote controller 7. The RS-232C I/F 21c is connected to the CCU 17, the endoscope unit 15 and the remote controller 7. As a result it is possible to send operating instructions to the CCU 17 and the endoscope unit 15 and to control them by the remote controller 7.

The USB I/F 21b is an interface for connecting the control unit 6 and the personal computer 25 electrically. In the case where the control unit 6 and the personal compute r 25 are connected via this USB I/F 21b, it is also possible to send instructions for displaying the endoscope image, and a range of control instructions such as for image processing while performing measurements, and the like, to the control unit 6 from a personal computer 25. Furthermore, it is also possible to input and output control information, data, and the like, as required for a range of processing between the control unit 6 and the personal computer 25.

An external memory medium such as a PCMCIA memory card 26, a compact flash (trademark) memory card 27, and the like, may be removably installed in the PC card I/F 21a. In the case where an external memory medium is installed, it is possible to input data such as control processing information, image information, and the like, stored in the external memory medium, to the control unit 6 via the PC card I/F 21a under control of the CPU 18. Alternatively, it is possible to supply data such as control processing information, image information, and the like, to the external memory medium via the PC card I/F 21a, for storage.

The video signal processing circuit 23 has a function for displaying a combined image comprising the endoscope image supplied from the CCU 17 and an operation menu displayed graphically. The video signal processing circuit 23 combines a video signal from the CCU 17, and a display signal of the operation menu generated by the CPU 18. Furthermore, the video signal processing circuit 23 supplies the LCD 8 after applying processing as required for display on the screen of the LCD 8. In this manner, the combined image of the endoscope image and the operation menu is displayed on the LCD 8. The video signal processing circuit 23 can also perform processing to display only the endoscope image, or an image such as the operation menu, independently.

The external image input terminal 11 for inputting an image to the video signal processing circuit 23 without passing through the CCU 17 is provided separately in the control unit 6. In the case where a video signal is input to this external image input terminal 11, the video signal processing circuit 23 outputs a combined image based on the video signal in precedence to the endoscope image from the CCU 17.

A sound signal collected by a microphone 28, and stored in the external storage medium, a sound signal obtained by reproduction from the external storage medium, and a sound signal generated by the CPU 18, are supplied to the sound signal processing circuit 22. Then, after the sound signal processing circuit 22 applies processing (amplification processing or the like) as required to reproduce the supplied sound signals, it outputs them to a speaker 22a. In this manner, sound signals are reproduced from the speaker 22a.

A joystick, a lever switch, a freeze switch, a store switch, a measurement execution switch, and the like, which are not shown in the figure, are provided on at least the top surface of the remote controller 7, so that a range of remote control operations can be performed.

Next is a detailed description of the stereo measurement optical adaptor 2 of the present embodiment, and the tip section 3a of the endoscope insertion section 3 to which it is connected. The present embodiment shows the case where an IC chip is used as an identification device in the stereo measurement optical adaptor 2.

As shown in FIG. 3, the tip section 3a becomes a connecting section 31 to which the stereo measurement optical adaptor 2 (referred to hereunder as simply optical adaptor 32) is connected. That is, the optical adaptor 32 is secured to the connecting section 31, by screwing a screw 33a on the proximal end of the optical adaptor 32 onto a mounting screw 33 on the connecting section 31.

An imaging unit 34 is provided on the connecting section 31. The imaging unit 34 is connected to the CCU 17 via a CCD cable 35. A CCD (light receiving section) 36, being an imager, is provided in the imaging unit 34. This CCD 36 is connected to the CCD cable 35 via a matching circuit 37. Furthermore, a light guide 38 (referred to hereunder as LG 38), connected to the light source 16, is provided in the connection section 31.

At the other side, an optical observation system (objective lens) 39 is provided in an area corresponding to (location facing) the CCD 36. Thus it is possible to form an image of the observed image on the light receiving surface of the CCD 36. Furthermore, an illumination light system 40 is provided in an area corresponding to the LG 38 of the optical adaptor 32. The optical adaptor 32 turns the light supplied from the light source 16 via the LG 38 into a light beam suitable for observation, to illuminate an object to be observed. Here, since the optical adaptor 32 of the present embodiment is a stereo measurement optical adaptor capable of viewing an object to be observed stereoscopically, it has two sets of optical observation systems 39. However, in the following description, only one set is used for simplicity of description.

As shown in FIG. 4, an identification IC chip 41, for identifying itself to the endoscope device 1 by which it is used, is integrated in the optical adaptor 32. This identification IC chip 41 is fixed in the optical adaptor 32 such that it is enclosed by a support material 42 formed from a non-metallic substance such as epoxy resin or the like. This identification IC chip 41 has an antenna for transmitting and receiving signals as well as for receiving energy for operation. This identification IC chip 41 is an IC having a 128 bit ROM as a data memory medium, and operates from a high frequency signal of 2.45 GHz, for example.

At the other side, an antenna 43 is provided in the area aligned with the identification IC chip 41 on the connecting section 31 side. The antenna 43 is connected to an identification circuit 51 described later, via an antenna line 44. The antenna line 43 and the CCD cable 35 are guided to the connecting section 31 through the endoscope insertion section 3.

FIG. 4 is a cross-sectional diagram showing a different cross section from FIG. 3, but the optical observation system 39 is also shown so that its location can be easily understood.

FIG. 5 is a view of the contact surface of the optical adaptor 32 and the connecting section 31, viewed from the connecting section 31 side. The identification IC chip 41 is fixed in the support material 42 beside the optical observation system 39 and the illumination light system 40. The support material 42 is elliptical, and the identification IC chip 41 is located near to one of the focal points of the ellipse. At the other side, the antenna 43 is provided in a location where it makes contact with the identification IC chip 41 as shown in FIG. 4.

The identification circuit 51 is a transmission and reception circuit 52 as shown in FIG. 6. This transmission and reception circuit 52 is connected to the CPU 18. Furthermore, this transmission and reception circuit 52 is connected to the antenna 43 via the antenna line 44.

Hereunder is a description of a stereo measurement method using the endoscope device 1 of the present embodiment with the construction described above.

This stereo measurement is performed by performing at least the following first to sixth processes. The first process reads identification information (ID), optical data (optical characteristic information) and the like, stored in the identification IC chip 41 of the optical adaptor 32 (stereo measurement optical adaptor 2). The second process reads information related to the relative position between the CCD 36 and the observation optical system 39 when the optical adaptor 32 is mounted on the tip section 3a of the endoscope insertion section 3. The third process obtains the positional error between the CCD 36 and the observation optical system 39 using the above described relative position information and information related to the relative position between the CCD (imager of the endoscope device, serving as a master) and the observation optical system 39 obtained when this optical adaptor 32 is mounted on the endoscope device, serving as a master, at the time of manufacture. The fourth process corrects the optical data using the positional error. The fifth process performs coordinate transformation of the measurement image based on the optical data after correction. The sixth process obtains three dimensional coordinates of an arbitrary point by matching the two images obtained by coordinate transformation.

The first process through the fourth process are collectively called the calibration process.

The CPU 18 performs the calibration process for the optical adaptor 32 once. Then, the CPU 18 performs control for storing the optical data after correction, which is obtained as a result, in the external storage medium (PCMCIA memory card 26, compact flash (trade mark) memory card 27, or the like) as measurement environment data. At this time, data related to the date and time when the calibration process is performed is stored as a part of the measurement environment data. In the case where stereo measurement is performed after t his calibration process is performed, the CPU 18 performs the fifth and the sixth processes by loading the measurement environment data from the external storage medium into the RAM 20.

Here, the second process is performed by obtaining the shape and location of a mask (not shown in the figure) provided in the optical adaptor 32. That is, it is performed by inserting the tip section 3a on which the optical adaptor 32 is mounted in a calibration tool 5 incorporating a white space, and capturing the blank image using the CCD 36.

In the measurement after the calibration process is performed, firstly the endoscope device 1 is switched on, and light from the light source 16 is guided to the tip section 3a via the LG 38. Light from the LG 38 is radiated on an object to be observed from an illuminating lens 40 in the optical adaptor 32. Returned light reflected from the object to be observed, that is the observed image, is formed on the CCD 36 via the observation optical system 39. The observed image is converted to an electrical signal by the CCD 36, and transmitted to the CCU 17 through the matching circuit 37 and the CCD cable 35. In the CCU 17, the electrical signal from the CCD 36 is converted to a normal video signal. Here, the electrical signal require d to operate the CCD 36 is generated by the video signal processing circuit 23, and supplied to the CCD 36 via the CCD cable 35.

FIG. 7 shows the exchange of data between the identification IC chip 41 and the CPU 18 by the transmission and reception circuit 52. This transmission and reception circuit 52 is connected to the CPU 18 by a bidirectional communication line as shown in FIG. 6. After the transmission signal generated by the CPU 18 is modulated at a high frequency, the transmission and reception circuit 52 transmits it to the antenna 43 of the connecting section 31 via the antenna line 44.

On receiving the transmission signal, the antenna 43 transmits an electromagnetic signal to the identification IC chip 41. By this electromagnetic signal reaching the identification IC chip 41, an instruction from the CPU 18 is transmitted. An ID (identification number) inquiry as shown in FIG. 7 is completed in this manner.

At this time, since the identification IC chip 41 is surrounded by the epoxy resin support material 42, the electromagnetic signal reaches the identification IC chip 41 satisfactorily. Since the support material 42 is elliptical, and the identification IC chip 41 is installed off center, then even if the wall on one side of this identification IC chip 41 is thin, it is possible for the electromagnetic signal to reach satisfactorily through the thick wall on the other side.

When an ID inquiry is received from the CPU 18, the identification IC chip 41 transmits the ID as return data. That is, the transmission data from the identification IC chip 41 is momentarily transmitted to the transmission and reception circuit 52 via the reverse route. This transmission data is demodulated by the transmission and reception circuit 52, and then transmitted to the CPU 18. Thus the ID reply as shown in FIG. 7 is completed.

Optical data stored by the identification IC chip 41 is also fetched by the CPU 18 using a similar procedure. That is, firstly the CPU 18 generates a transmission signal for an optical data inquiry. Then the transmission and reception circuit 52 modulates it at a high frequency, and afterwards transmits it to the antenna 43 of the connecting section 31 via the antenna line 44.

On receiving the transmission signal, the antenna 43 transmits an electromagnetic signal to the identification IC chip 41. By this electromagnetic signal reaching the identification IC chip 41, an instruction from the CPU 18 is transmitted. An optical data inquiry as shown in FIG. 7 is completed in this manner.

Then, on receiving an optical data inquiry from the CPU 18, the identification IC chip 41 transmits the optical data as return data. That is, the transmission data from the identification ID chip 41 is transmitted to the transmission and reception circuit 52 via the reverse route. This transmission data is demodulated by the transmission and reception circuit 52, and then transmitted to the CPU 18. Thus the series of communication related to optical data as shown in FIG. 7 is completed.

Even in the case where there is data other than the ID and optical data to be read, it is fetched by the same procedure.

FIG. 8 shows an example of the format of communication data exchanged between the identification IC chip 41 and the CPU 18. An ID inquiry contains the two letter data ‘ID’ transmitted from the CPU 18 to the identification IC chip 41. Here, (EOF) is a delimiting symbol indicating the end of data. Furthermore, an optical data inquiry contains the four letter data ‘DATA’. The ID data transmitted from the identification ID chip 41 to the CPU 18 is a four digit number, and is the same number as the number marked on the outside of the optical adaptor 32. The optical data transmitted from the identification IC chip 41 to the CPU 18 are the number 120, indicating the angle of view, and two three digit numbers indicating the x coordinate and the y coordinate of the center of the screen, delimited by commas.

The optical data transmitted from the identification IC chip 41 to the CPU 18 are used by the CPU 18 for calculation at the time of measurement, and are optical characteristics unique to each optical adaptor, expressed as constant numbers. For this optical data, there are four points from (a) to (d) as described in paragraph number (0014) of Japanese Unexamined Patent Application, First Publication No. H10-248806. To be specific, (a) geometric distortion correction tables for the two optical systems, (b) the focal lengths of the two lens systems, (c) the distance between the optical axes of the two lenses, and (d) positional information of the two images of a master. There is a possibility that additional elements are contained in the optical data. However, the operation of reading from the identification IC chip 41 to the CPU 18 is the same as above.

Furthermore, the detail of coordinate transformation calculation (distortion correction calculation) of an image by this optical data is described in equations (1) and (2) of the above-described Japanese Patent Publication.

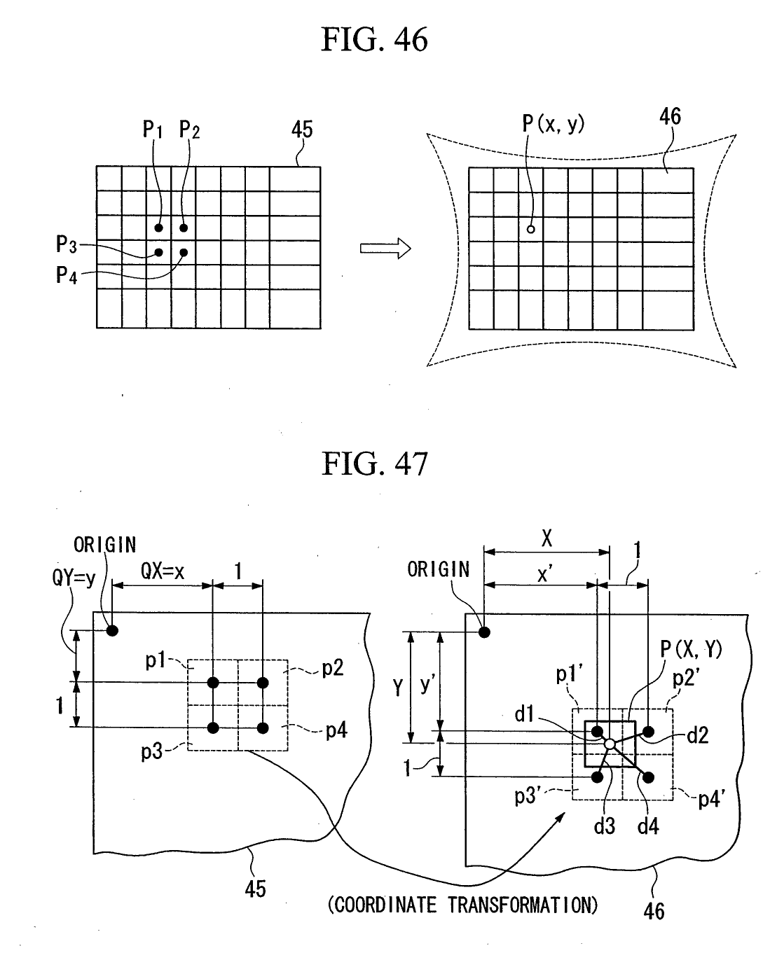

Next is a detailed description of each piece of optical data unique to the above-described optical adaptor 32. Regarding (a) the geometric distortion correction tables, in general, an image by a lens system has optical distortion. Since this distortion causes large errors when measurements are performed, it is removed by performing coordinate transformation. Coordinate transformation may be performed with the optical axis being in the center, or in the case where more accurate correction is performed, the center of geometric distortion of the optical system may be used. Furthermore, a geometric distortion correction table for the two images may be provided individually as a right image and a left image, or the two may be combined to make one table. Hereunder is a description of a correction table based on FIGS. 46 and 47 in the case of one table.

In FIGS. 46 and 47, points p1 to p4 on the imaging screen 45 show pixels before coordinate transformation. When the coordinates p1 to p4 are transformed by f (x, y), they become p1′ to p4′. The coordinates that give p1′ to p4′ at this time are not always integer numbers, and are obtained as the actual values of the coordinates. In order to display p1′ to p4′ on the screen 46 of a liquid crystal monitor after transformation, coordinates (X, Y) of the pixel P (X, Y) after transformation must be transformed to integer numbers in units of pixels.

The correction for creating the coordinate integer numbers is performed by weight tables W1 to W4. That is, considering optical geometric distortion, the pixel data P (X, Y) of a transformed screen pixel are obtained for each pixel on the transformed screen by multiplying the image data of the four pixels at the coordinates to which that pixel corresponds on the imaging screen by the ratios in the above-described weight tables W1 to W4.

Therefore, the coordinate transformation of f (x, y) uses the following equation to obtain transformed coordinates x′, y′.
x′=k1×(a₀₀+a₃₀x³+a₁₂xy²+a₅₀x⁵+a₃₂x³y²+a₁₄xy⁴+a₇₀x+a₅₂x⁵y²+a₃₄x³y⁴+a₁₆xy⁶) (1)
y′=k2×(b₀₀+b₂₁x²y+b₀₃y³+b₄₁x⁴y+b₂₃x²y³+b₀₅y⁵+b₆₁x⁶y+b₄₃x⁴y²+b₂₅x²y⁵+b₀₇y⁷) (2)

Here, the coefficients aₙₘ and bₙₘ are obtained from the linearity of the lattice image. Furthermore, k1 and k2 are coefficients matching the magnification of the two images, and the functions of focal lengths fR and fL.

The coordinates (x′, y′), which describe p1′ (x′, y′) to p4′ (x′, y′), are obtained by substituting the x, y coordinates of p1 to p4 in the above f (x, y). The values of x′ and y′ are not always integer numbers as mentioned above, and the pixel data for the coordinates (X, Y) after transformation to integer numbers are obtained by correction using the weight tables W1 to W4.

Therefore, the coordinates of the four points p1′ to p4′ on the transformed screen, which give pixel data P (X, Y) after transformation, are (x′, y′), and the x coordinate of the coordinates (x, y) of the top left point p1 among the coordinates (x, y) to (x+1, y+1) of the four points on the imaging screen (original picture) corresponding to the four points p1′ to p4′ on the transformed screen, are QX (X, Y), and the y coordinate is QY (X, Y). They are stored firstly to the identification IC chip 41 as a geometric distortion correction coordinate reference table.

Pixel data P (X, Y) after transformation of the coordinates (X, Y), which are given as integer numbers in pixel units after transformation, can be obtained using p1′ to p4′ and W1 to W4.

Where, as shown in FIG. 7, dn denotes the distance between p1′ to p4′ and P (X, Y),
S=d1+d2+d3+d4 (3)
furthermore,
W1=d1/S
W2=d2/S
W3=d3/S
W4=d4/S (4).

The value of P (X, Y) can be obtained by
P(X,Y)=W1×p1′+W2×p2′+W3×p3′+W4×p4′ (5)

The above-described W1, W2, W3 and W4 are stored in the identification IC chip 41 with the coordinate reference tables QX (X, Y) and QY (X, Y) as weight tables for each pixel point (X, Y) on each transformed screen.

Next, regarding (b) to (d),

(b) The focal length fR of the right image, and the focal length fL of the left image, are measured and stored as the focal lengths of the two lens systems.

(c) The optical axis coordinates XR and YR of the right image, and the optical axis coordinates XL and YL of the left image, are measured and stored as the optical axis coordinates of the two lens systems.

(d) The brightness data PV (100, Yn) of a reference line V (vertical line), where Yn=1, 2, 3, . . . 480, and the brightness data PH (Xn, 100) of a reference line H (horizontal line), where Xn=1, 2, 3, . . . 640, are measured and stored as positional information (visual shape pattern) of a master.

According to the endoscope device 1 of the present embodiment described above, it is possible to obtain the following effects.

The endoscope device 1 of the present embodiment uses a construction wherein its optical adaptor 32 incorporates an identification IC chip 41 in which optical data of the observation optical system 39 is stored, and an antenna 43 is provided in the tip section 3a of the endoscope insertion section 3. Using this construction, it is possible to automate the operation of identifying the optical adaptor 32 without confirmation operations by the user. Accordingly, it is possible to identify the optical adaptor to be used accurately, thus enabling misoperation by the user to be prevented.

That is, in the endoscope device 1 of the present embodiment, since the optical characteristic values of the optical adaptor 32 to be used are held in the optical adaptor 32, it is not necessary to hold the optical data of the optical adaptor 32 on the control unit 6 side in advance. Accordingly, provided with only the identification IC chip 41, a calibration process for the registration and selection of optical data can be performed automatically with any optical adaptor. Once registered, from the next time it is possible to load corresponding environmental data into the RAM 20 by only detecting the ID. Thus it is possible to perform measurements immediately.

Furthermore, the endoscope device 1 of the present embodiment uses a construction wherein exchange of information between the identification IC chip 41 and the CPU 18 is performed without contact using radio communication. Using this construction, electrical contact points are not required on the optical adaptor 32 side, which enables easy assembly. Furthermore, the non-contact system enables higher durability to be ensured than a contact system.

(Second Embodiment)

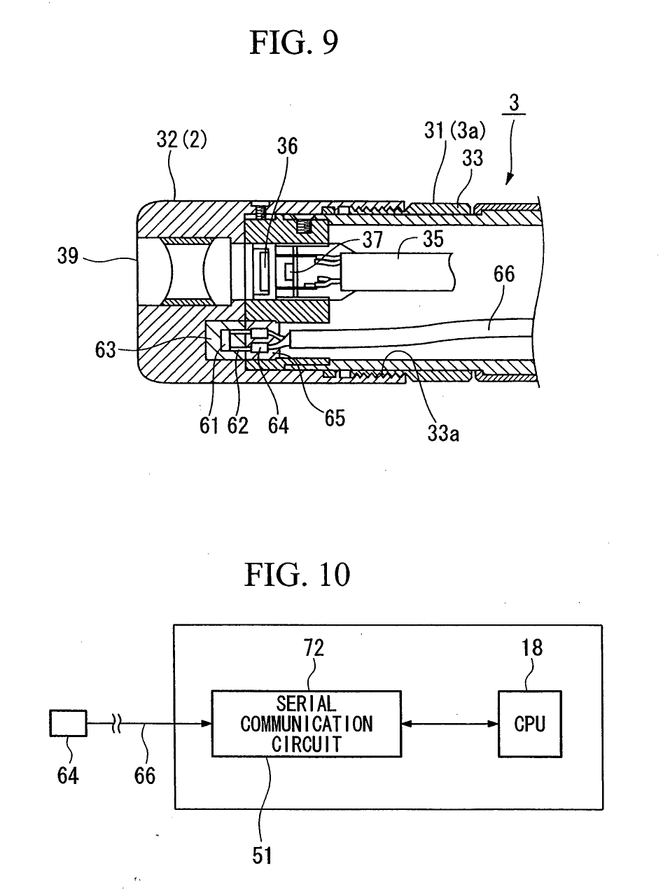

Hereunder is a description of a second embodiment of the present invention with reference to FIG. 9 and FIG. 10. FIG. 9 is a diagram showing the main parts of an endoscope device of the present embodiment, being a cross-sectional diagram showing the tip section of the endoscope insertion section, and an optical adaptor installed therein. Furthermore, FIG. 10 is a block diagram of an electrical circuit provided in the endoscope device.

In the following description, the description focuses on the differences from the first embodiment, and the same symbols are used for the same structural elements as in the first embodiment, and their descriptions are omitted.

The distinguishing characteristic of the present embodiment is that information exchange between the identification IC chip 41 and the CPU 18 is performed by mechanical contact rather than the non-contact system in the first embodiment.

That is, as shown in FIG. 9, the identification IC chip (in the description hereunder, a new numeral 61 is assigned in order to distinguish it from the identification IC chip 41) is provided with a pair of IC side contact points 62 fixed in a support material 63 formed from epoxy resin. Furthermore, this identification IC chip 61 incorporates a CPU, which contains internal ROM and RAM. This identification IC chip 61 communicates with the outside using energy supplied from the communication line on the control unit 6, and also has a function of supplying optical information required for a calibration process to the outside.

At the other side, a pair of endoscope side contacts 64 is fixed in a contact support material 65 formed from epoxy resin on the connecting section 31 (tip section 3a) of the endoscope insertion section 3. The pair of endoscope side contacts 64 transmits an electrical signal by making contact with each of the IC side contacts 62 of the identification IC chip 61 provided on the optical adaptor 32 (stereo measurement optical adaptor 2) side. The endoscope side contacts 64 are connected to the CCU 17 via a two core communication line 66.

Moreover, in the present embodiment, as shown in FIG. 10, a serial communication circuit 72 is used as the identification circuit 51 instead of the transmission and reception circuit 52. This serial communication circuit 72 transmits a communication signal from the CPU 18 to each of the endoscope side contacts 64 via the two core communication line 66. Furthermore, the communication signal is transmitted to the identification IC chip 61 via both of the IC side contacts 62 connected to the endoscope side contacts 64. Conversely, the communication signal from the identification IC chip 61 to the CPU 18 is transmitted via the reverse route.

In the endoscope device 1 of the present embodiment having the above-described construction, the IC side contacts 62 are connected to the respective endoscope side contacts 64 mechanically by mounting the optical adaptor 32 on the tip section 3a. Thus the connection is completed automatically. The flow of the calibration process performed after this is almost the same as the flow described in the first embodiment.

According to the endoscope device 1 of the present embodiment as described above, it is possible to obtain the same effect as in the first embodiment. That is, it is possible to automate the operation of identifying the optical adaptor 32 (stereo measurement optical adaptor 2) without confirmation operations by the user. Accordingly, it is possible to identify the type of optical adaptor 32 to be used accurately, thus enabling misoperation by the user to be prevented.

Furthermore, the endoscope device 1 of the present embodiment uses a construction in which communication data from the identification IC chip 61 to the CPU 18 is read via the connection between the IC side contacts 62 and the endoscope side contacts 64. Using this construction, since communication data is read via mechanical contacts, it is possible to use a relatively large IC chip for the identification chip 61 compared with the case of wireless. In this manner, it is possible to increase the amount of data held on the optical adaptor 32 side.

(Third Embodiment)

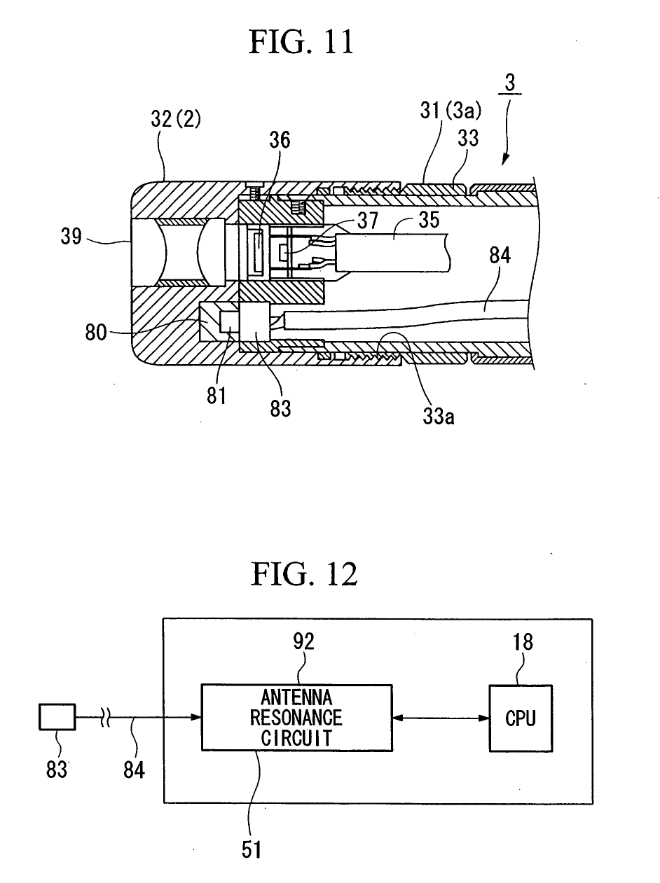

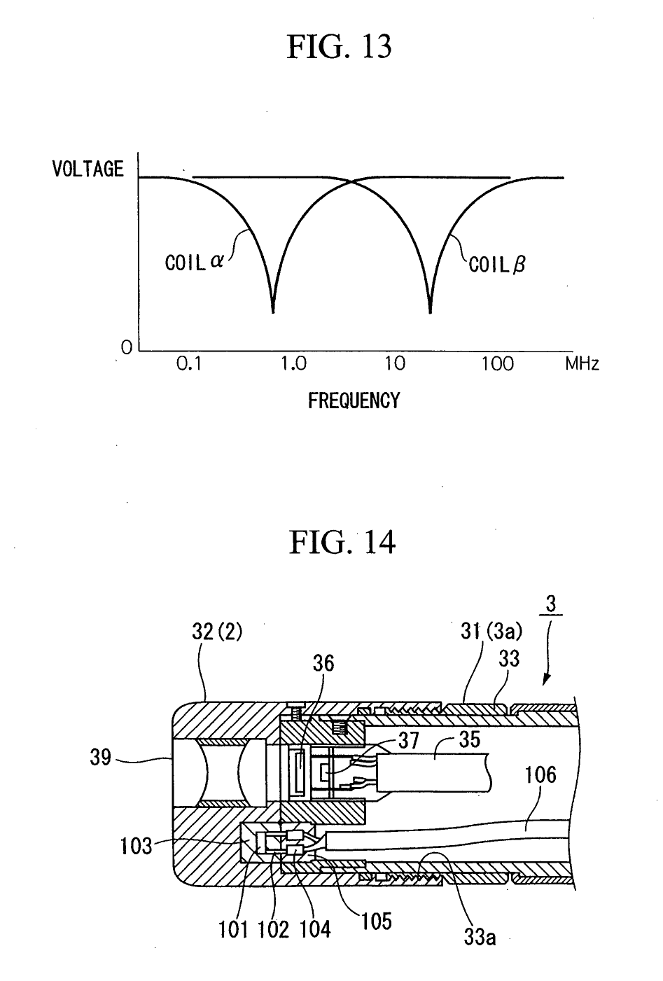

Hereunder is a description of a third embodiment of the present invention with reference to FIG. 11 to FIG. 13. FIG. 11 is a diagram showing the main parts of an endoscope device 1 of the present embodiment, being a cross-sectional diagram showing the tip section 3a of the endoscope insertion section 3, and an optical adaptor 32 installed therein. FIG. 12 is a block diagram of an electrical circuit provided in the endoscope device 1. Furthermore, FIG. 13 is a graph showing the voltage of the electrical circuit of the endoscope device 1, wherein the horizontal axis shows frequency, and the vertical axis shows voltage.

In the following description, the description focuses on the differences from the first embodiment, and the same symbols are used for the same structural elements as in the first embodiment, and their descriptions are omitted.

The distinguishing characteristic of the present embodiment is that a combination of high frequency coils is used, instead of the combination of the identification IC chip 41 and the antenna 43 in the first embodiment, and optical adaptors 32 (stereo measurement optical adaptor 2) installed are identified by different resonance frequencies generated when they resonate.

That is, as shown in FIG. 11, a coil 81 fixed in a support material 80 formed from epoxy resin is incorporated in the optical adaptor 32, instead of the identification IC chip 41.

At the other side, an antenna coil 83 is provided at a location in the connecting section 31 (tip section 3a) of the endoscope insertion section 3 aligned with the coil 81 when the optical adaptor 32 is connected to the tip section 3a. This antenna coil 83 is connected to the CCU 17 via an antenna line 84 as shown in the figure.

Furthermore, in the present embodiment, as shown in FIG. 12, an antenna resonance circuit 92 is used as the identification circuit 51 instead of the transmission and reception circuit 52. This antenna resonance circuit 92 excites the antenna coil 83 at a predetermined frequency when it receives an instruction from the CPU 18. At the same time, the antenna resonance circuit 92 monitors the voltage at this time, and sends the voltage back to the CPU 18.

As shown in FIG. 13, in the case where a coil α with a high inductance is used as the coil 81, the resonance frequency is low. Conversely, in the case where a coil β with a low inductance is used as the coil 81, the resonance frequency is high. Accordingly, by checking the voltage as it increases and decreases depending on the height of the resonance frequency, it is possible to distinguish the type of optical adaptor 32 connected. The identification operation is performed using the same operations as in the so-called dipmeter principle.

To explain the operation of identifying this optical adaptor 32 with a specific example, firstly, the CPU 18 sends an instruction for an antenna resonance circuit 92 to excite the antenna coil 83 at 0.1 MHz, for example. Then, the antenna resonance circuit 92 excites the antenna coil 83 at 0.1 MHz, and also sends the voltage generated at that time back to the CPU 18. The CPU 18 stores the voltage, and sends an instruction to excite at 0.2 MHz this time. Changing the excitation frequency in this manner to 0.33 MHz, 0.35 MHz, 0.7 MHz, 1 MHz up to 700 MHz sequentially, the voltages are stored at each of the frequencies.

Subsequently, the CPU 18 searches the results for the frequency at which the voltage is highest, to determine the resonance frequency. The resonance frequency obtained in this manner can act as an identifier for identifying the optical adaptor 32 installed. Accordingly, by providing the type of the optical adaptor 32 corresponding to the resonance frequency, and its optical data, on the control unit 6 side in advance (providing it in the external storage medium), it is possible to select the optical data required for the calibration process. The calibration process performed after this is almost the same as the flow described in the first embodiment.

According to the endoscope device 1 of the present embodiment as described above, it is possible to obtain the same effect as in the first embodiment. That is, it is possible to automate the operation of identifying the optical adaptor 32 (stereo measurement optical adaptor 2) without confirmation operations by the user. Accordingly, it is possible to identify the type of optical adaptor 32 used accurately, thus enabling misoperation by the user to be prevented.

Furthermore, the endoscope device 1 of the present embodiment uses a construction in which the operation of identifying the optical adaptor 32 is performed by determining the resonance frequency generated between the coil 81 and the antenna coil 83. Using this construction, it is not necessary to use electrical contact points, it being only necessary to provide the coil 81 in the optical adaptor 32. Thus it is possible to assemble it easily. Moreover, it is possible to obtain information by a non-contact system, which also enables higher durability to be ensured than a contact system.

(Fourth Embodiment)

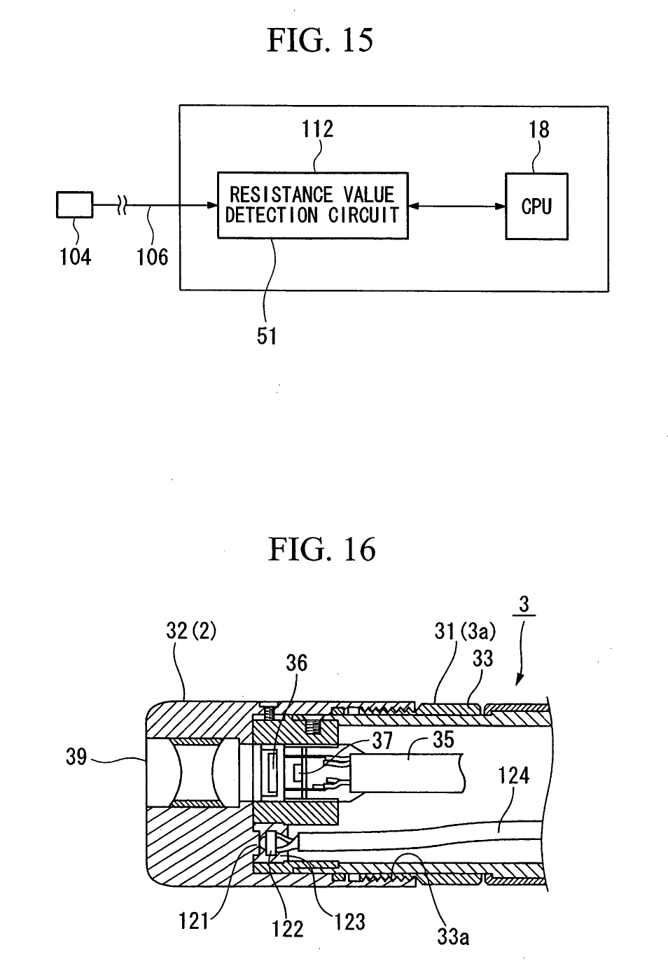

Hereunder is a description of a fourth embodiment of the present invention with reference to FIG. 14 and FIG. 15. FIG. 14 is a diagram showing the main parts of an endoscope device 1 of the present embodiment, being a cross-sectional diagram showing the tip section 3a of the endoscope insertion section 3, and an optical adaptor 32 installed therein. FIG. 15 is a block diagram of an electrical circuit provided in the endoscope device 1.

In the following description, the description focuses on the differences from the first embodiment, and the same symbols are used for the same structural elements as in the first embodiment, and their descriptions are omitted.

The distinguishing characteristic of the present embodiment is that a resistor is used instead of the identification IC chip 41 in the first embodiment, and the type of optical adaptor 32 is identified by obtaining the value of its resistance.

That is, as shown in FIG. 14, an identification resistor 101 is incorporated in the optical adaptor 32 instead of the identification IC chip 41. This identification resistor 101 has a pair of resistor side contacts 102 fixed in a support material 103 formed f rom epoxy resin.

At the other side, a pair of endoscope side contacts 104 is provided on the connection section 31 (tip section 3a) of the endoscope insertion section 3. The pair of endoscope side contacts 104 is connected to the identification resistor 101 when the optical adaptor 32 is connected to the tip section 3a, and transmits an electrical signal. The endoscope side contacts 104 are fixed in the connecting section 31 by a support material 105 formed from epoxy resin, and connected to the CCU 17 via a communication line 106 as shown in the figure.

Furthermore, in the present embodiment, as shown in FIG. 15, a resistance value detection circuit 112 is used as the identification circuit 51 instead of the transmission and reception circuit 52. This resistance value detection circuit 112 supplies a predetermined (constant) current to the identification resistor 101 via the communication line 106, and also has a function of sending the value of the voltage generated at that time to the CPU 18.

The voltage obtained at this time can act as an identifier for identifying the optical adaptor 32 installed. Accordingly, by providing the type of the optical adaptor 32 corresponding to the voltage value, and its optical data, on the control unit 6 side in advance (providing it in the external storage medium), it is possible to select the optical data required for the calibration process. The calibration process performed after this is almost the same as the flow described in the first embodiment.

According to the endoscope device 1 of the present embodiment as described above, it is possible to obtain the same effect as in the first embodiment. That is, it is possible to automate the operation of identifying the optical adaptor 32 (stereo measurement optical adaptor 2) without confirmation operations by the user. Accordingly, it is possible to identify the type of optical adaptor 32 used accurately, thus enabling misoperation by the user to be prevented.

Moreover, the endoscope device 1 of the present embodiment uses a construction in which the operation of identifying the optical adaptor 32 is performed by reading a voltage value determined by the resistance value of the identification resistor 101. Using this construction, it is possible to set the value of resistance of the identification resistor 102 finely. Thus the construction may be such that the optical adaptors 32 can be identified easily even if there is a large number of types of them.

(Fifth Embodiment)

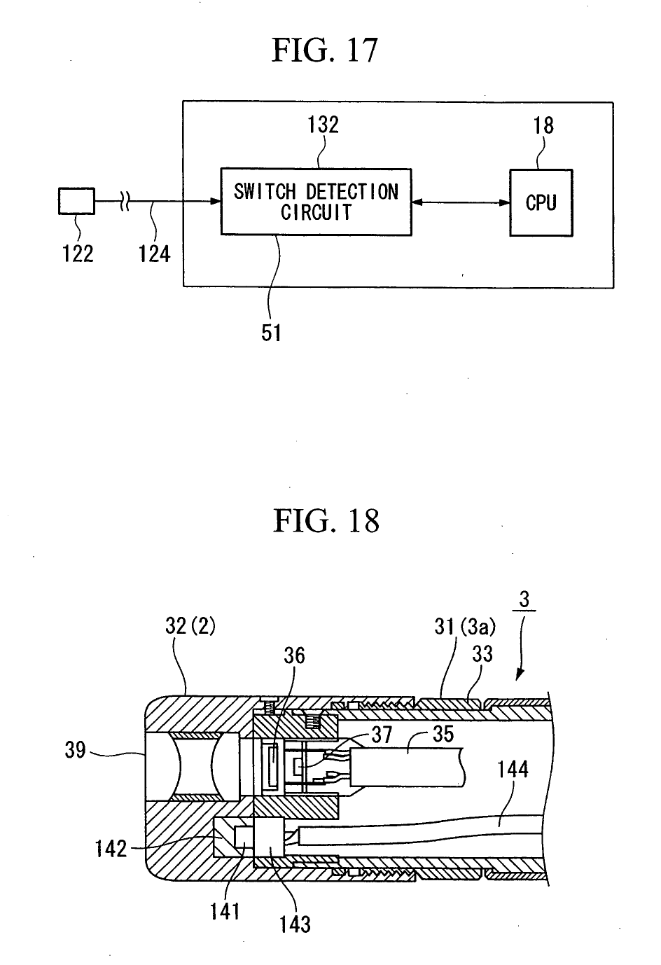

Hereunder is a description of a fifth embodiment of the present invention with reference to FIG. 16 and FIG. 17. FIG. 16 is a diagram showing the main parts of an endoscope device of the present invention, being a cross-sectional diagram showing the tip section of the endoscope insertion section, and an optical adaptor installed therein. Furthermore, FIG. 17 is a block diagram of an electrical circuit provided in the endoscope device 1.

In the following description, the description focuses on the differences from the first embodiment, and the same symbols are used for the same structural elements as in the first embodiment, and their descriptions are omitted.

The distinguishing characteristic of the present embodiment is that a mechanical switch is used for determining the optical adaptor 32, in contrast to the first embodiment.

That is, as shown in FIG. 16, the optical adaptor 32 of the present embodiment is provided with an identification projection 121 that protrudes toward, and connects with, the connecting section 31.

At the other side, an identification switch (mechanical switch) 122 is fixed in a switch support material 123 formed from epoxy resin on the connecting section 31 (tip section 3a) of the endoscope insertion section 3. The identification projection 121 makes contact with the identification switch 122 when the optical adaptor 32 is connected to the tip section 3a. This identification switch 122 is connected to the CCU 17 via a signal line 124 as shown in the figure. Here, only one identification switch 122 is shown in the figure. However, two are provided in a real situation.

Furthermore, in the present embodiment, as shown in FIG. 17, a switch detecting circuit 132 is used as the identification circuit 51 instead of the transmission and reception circuit 52. This switch detecting circuit 132 has a function of transmitting ON/OFF signals from the identification switch 122 to the CPU 18. Since two identification switches 122 are provided, it is possible to determine four states by the combination of ON/OFF signals. However, in practice, one of them is a state in which an optical adaptor 32 is not installed, so it is eliminated. Thus it is possible to identify three types of optical adaptor 32.

Accordingly, the combinations of ON/OFF signals obtained in this manner can act as an identification number for identifying the optical adaptor 32 installed. Therefore, by providing the type of the optical adaptor 32 corresponding to the ON/OFF signals, and its optical data, on the control unit 6 side in advance (providing it in the external storage medium), it is possible to select the optical data required for the calibration process. The calibration process performed after this is almost the same as the flow described in the first embodiment.

According to the endoscope device 1 of the present embodiment as described above, it is possible to obtain the same effect as in the first embodiment. That is, it is possible to automate the operation of identifying the optical adaptor 32 (stereo measurement optical adaptor 2) without confirmation operations by the user. Accordingly, it is possible to identify the type of optical adaptor 32 used accurately, thus enabling misoperation by the user to be prevented.

Moreover, in the endoscope device 1 of the present embodiment, only the identification projections 121 need to be provided on the optical adaptor 32 side, which enables it to be used easily and with low cost.

(Sixth Embodiment)

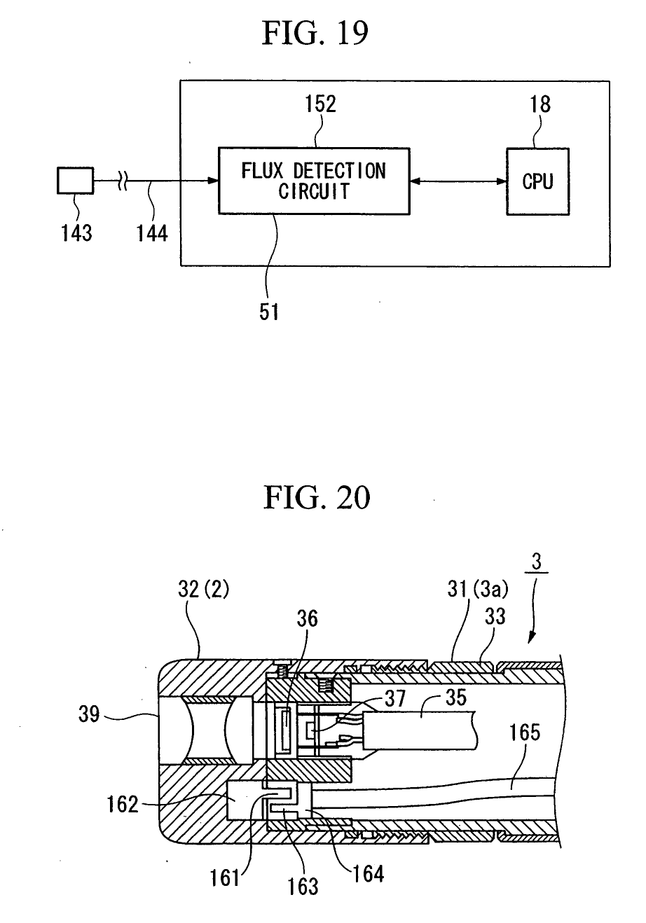

Hereunder is a description of a sixth embodiment of the present invention with reference to FIG. 18 and FIG. 19. FIG. 18 is a diagram showing the main part s of an endoscope device 1 of the present invention, being a cross-sectional diagram showing the tip section 3a of the endoscope insertion section 3, and an optical adaptor installed therein. Furthermore, FIG. 19 is a block diagram of an electrical circui t provided in the endoscope device 1.

In the following description, the description focuses on the differences from the first embodiment, and the same symbols are used for the same structural elements as in the first embodiment, and their descriptions are omitted.

The distinguishing characteristic of the present embodiment is that a combination of a magnet 141 and a hall element 143 is used, instead of the combination of the identification IC chip 41 and the antenna 43 in the first embodiment, and the type of optical adaptor 32 is identified by obtaining the strength and polarity of the magnet 141.

That is, as shown in FIG. 18, the optical adaptor 32 of the present embodiment is provided with the magnet 141 fixed in a support material 142 formed from an epoxy resin of a non-magnetic material.

At the other side, the hall element 143 is fitted in a location on the connecting section 31 (tip section 3a) of the endoscope insertion section 3 aligned with the magnet 143 when the optical adaptor 32 is connected to the tip section 3a. This hall element 143 is connected to the CCU 17 via a connecting cable 144 as shown in the figure.

Moreover, in the present embodiment, as shown in FIG. 19, a flux detection circuit 152 is used as the identification circuit 51 ins tead of the transmission and reception circuit 52. This flux detection circuit 152 has a function of driving the hall element 143 and sending the flux level detected therein to the CPU 18. Accordingly, when the optical adaptor 32 is mounted on the connecting section 31, the flux density detected by the hall element 141 changes due to the magnetic field generated by the magnet 141. The flux density (strength and polarity of the magnet 141) obtained in this manner can act as an identifier for identifying the optical adaptor 32 installed. Accordingly, by providing the type of the optical adaptor 32 corresponding to the flux density, and its optical data, on the control unit 6 side in advance (providing it in the external storage medium), it is possible to select the optical data required for the calibration process. The calibration process performed after this is almost the same as the flow described in the first embodiment.

According to the endoscope device 1 of the present embodiment as described above, it is possible to obtain the same effect as in the first embodiment. That is, it is possible to automate the operation of identifying the optical adaptor 32 (stereo measurement optical adaptor 2) without confirmation operations by the user. Accordingly, it is possible to identify the type of optical adaptor 32 used accurately, thus enabling misoperation by the user to be prevented.

Furthermore, in the endoscope device 1 of the present embodiment, since it is not necessary to use electrical contact points, it can be assembled easily. Moreover, it is possible to obtain information by a non-contact system, thus enabling higher durability to be ensured than a contact system.

(Seventh Embodiment)

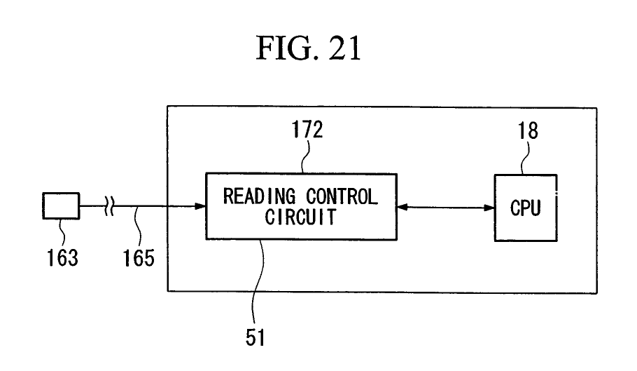

Hereunder is a description of a seventh embodiment of the pre sent invention with reference to FIG. 20 and FIG. 21. FIG. 20 shows the main parts of an endoscope device 1 of the present embodiment, being a cross-sectional diagram showing the tip section 3a of the endoscope insertion section 3, and an optical adaptor 32 installed therein. Furthermore, FIG. 21 is a block diagram of an electrical circuit provided in the endoscope device 1.

In the following description, the description focuses on the differences from the first embodiment, and the same symbols are used for the same structural elements as in the first embodiment, and their descriptions are omitted.

The distinguishing characteristic of the present embodiment is that a combination of a character/image information display section 161 and a receiving device 163 is used, instead of the combination of the identification IC chip 41 and the antenna 43 in the first embodiment, and the type of optical adaptor 32 is identified based on character/image information.

That is, as shown in FIG. 20, the character/image information display section 161, in which character/image information is written on the side face of an elongated rod shaped material in the optical adaptor 32 of the present embodiment, is fixed in a fixing material 162.

At the other side, the receiving device 163 is fitted in a location on the connecting section 31 (tip section 3a) of the endoscope insertion section 3 aligned with the character/image information display section 161 when the optical adaptor 32 is connected to the tip section 3a. This receiving device 163 is connected to the CCU 17 via a signal line 165 as shown in the figure.

Furthermore, in the present embodiment, as shown in FIG. 21, a reading control circuit 172 is used as the identification circuit 51 instead of the transmission and reception circuit 52. This reading control circuit 172 has a function of communicating with the receiving device 163, and sending the character/image information thus detected to the CPU 18. Accordingly, when the optical adaptor 32 is mounted on the connecting section 31, the character/image information display section 161 faces the receiving device 163, thus the receiving device 163 reads its character/image information, and converts it to a digital signal. Then, this digital signal is transmitted to the CPU 18 via the signal line 165.

The character/image information obtained in this manner can act as an identifier for identifying the optical adaptor 32 installed. Accordingly, by providing the type of the optical adaptor 32 corresponding to the character/image information, and its optical data, on the control unit 6 side in advance (providing it in the external storage medium), it is possible to select the optical data required for the calibration process. The calibration process performed after this is almost the same as the flow described in the first embodiment.

According to the endoscope device 1 of the present embodiment as described above, it is possible to obtain the same effect as in the first embodiment. That is, it is possible to automate the operation of identifying the optical adaptor 32 (stereo measurement optical adaptor 2) without confirmation operations by the user. Accordingly, it is possible to identify the type of optical adaptor 32 used accurately, thus enabling misoperation by the user to be prevented.

(Eighth Embodiment)

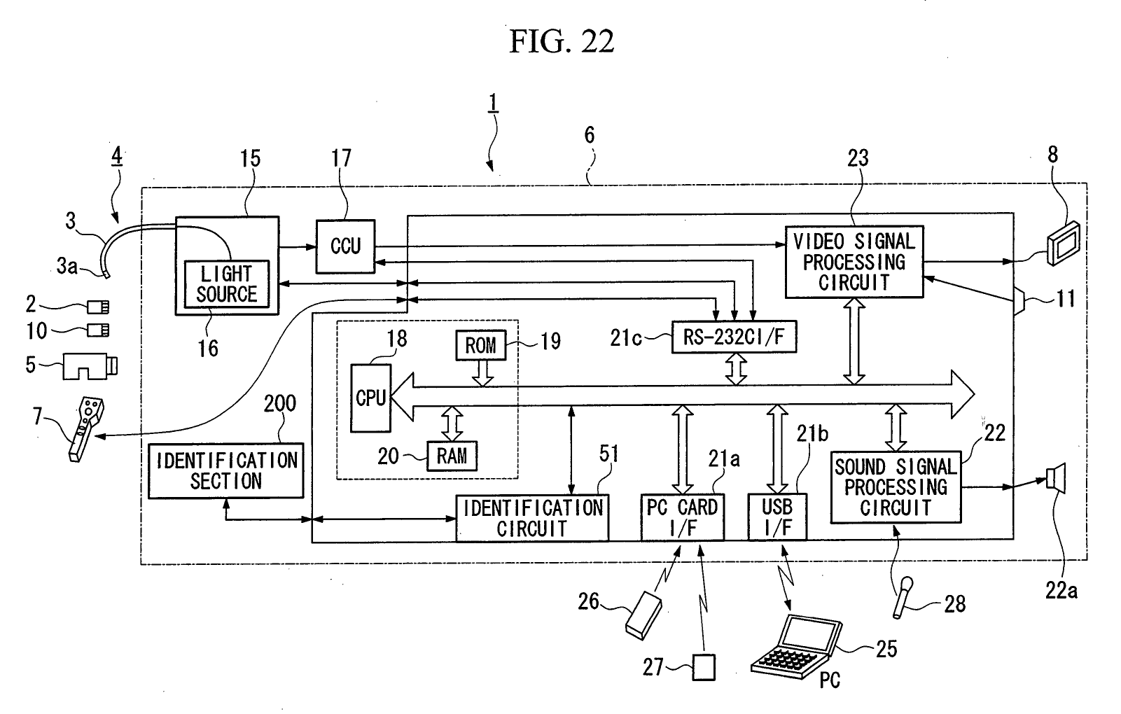

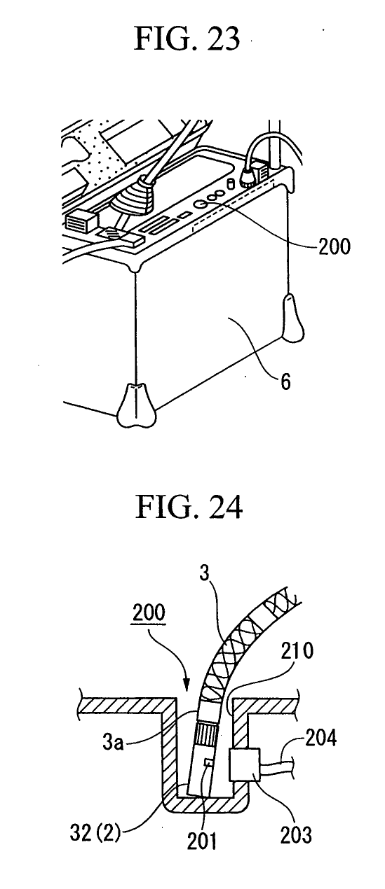

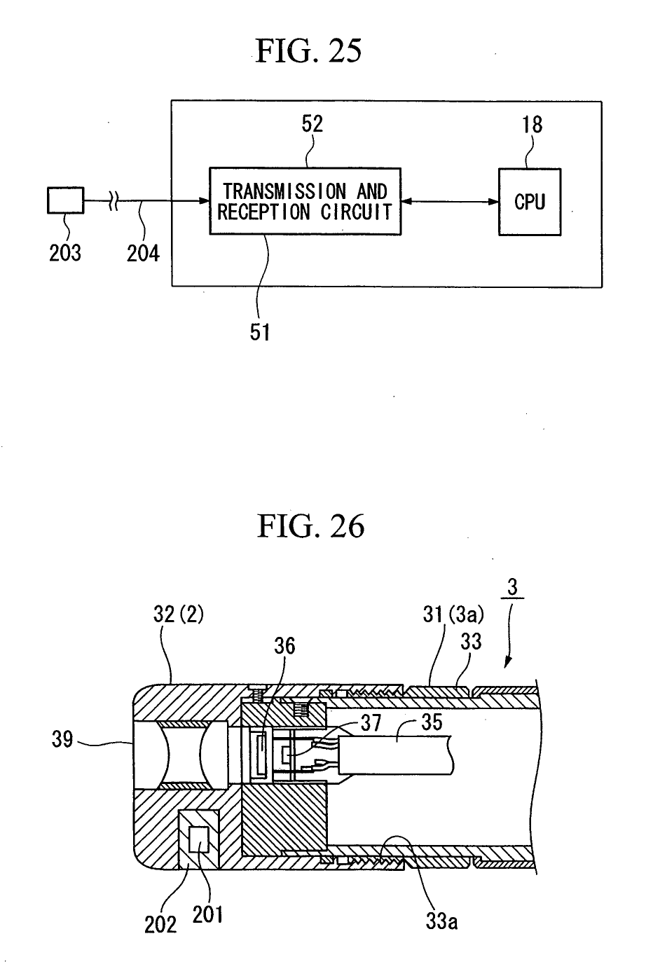

Hereunder is a description of an eighth embodiment of the present invention with reference to FIG. 22 to FIG. 27. FIG. 22 is a block diagram showing the internal structure of an endoscope device 1 of the present embodiment. FIG. 23 is a perspective view showing the location of an identification section provided in the endoscope device 1. FIG. 24 is a cross-sectional diagram showing the identification section. FIG. 25 is a block diagram of an electrical circuit provided in the endoscope device 1. FIG. 26 is a diagram showing the main parts of the endoscope device 1, being a cross-sectional diagram showing the tip section 3a of the endoscope insertion section 3, and an optical adaptor 32 installed therein.

In the following description, the description focuses on the differences from the first embodiment, and the same symbols are used for the same structural elements as in the first embodiment, and their descriptions are omitted.

In the first embodiment to the seventh embodiment, the identification section (the antenna 43, the endoscope side contacts 64, the antenna coil 83, the endoscope side contacts 104, the identification switch 122, the hall element 143 or the receiving device 163) is provided on the endoscope insertion section 3 side. However, the distinguishing characteristic of the present embodiment is that it is provided on the main body side (control unit 6 side) as shown in FIG. 22 to FIG. 24.

That is, as shown in FIG. 23 and FIG. 24, an identification section 200 is fitted in the panel of the control unit 6. The identification operation is performed by inserting the endoscope insertion section 3 on which the optical adaptor 32 is mounted into this identification section 200. The identification section 200 comprises a cavity 210, into which the optical adaptor 32 is inserted, and an antenna 203 provided in this cavity 210.

The antenna 203 is placed so as to line up with an identification IC chip 201 of the optical adaptor 32, which is inserted in the cavity 210, as shown in FIG. 24. Furthermore, this antenna 203 is connected to the identification circuit 51 via an antenna line 204 as shown in FIG. 25. In the present embodiment, the transmission and reception circuit 52 is used as this identification circuit 51.

However, the antenna 43 and the antenna line 44 are not provided on the connecting section 31 side. It is therefore possible to make a proportionate reduction to the outside diameter of the endoscope insertion section 3 including this connect ing section 31.

As shown in FIG. 26, the identification IC chip 201 for identifying itself to the endoscope device 1 by which it is used, is integrated in the optical adaptor 32.

This identification IC chip 201 has an antenna for receiving energy for ope ration, as well as for transmitting and receiving signals. This identification IC chip 201 is an IC having a 128 bit ROM as a data memory medium. For example, it operates from a high frequency signal of 2.45 GHz. This identification IC chip 201 is fixed in the optical adaptor 32 such that it is enclosed by a support material 202 formed from a non-metallic substance such as epoxy resin or the like. The support material 202 is elliptical, similarly to the support material 42, and the identification IC chip 201 is located near to one of the focal points of the ellipse.

In the endoscope device 1 of the present embodiment having the above construction, in the case where the optical adaptor 32 is changed or newly installed, it is possible to perform the calibration process automatically by doing nothing other than inserting the tip section 3a together with the optical adaptor 32.

That is, in the situation where the optical adaptor 32 is inserted in the cavity 210, firstly, the CPU 18 generates a transmission signal for an ID (identification number) inquiry. After modulating this at a high-frequency, the transmission and reception circuit 52 sends it to the antenna 203 of the identification section 200 via the antenna line 204.

On receiving the transmission signal, the antenna 203 sends an electromagnetic signal to the identification IC chip 201. By this electromagnetic signal reaching the identification IC chip 201, an instruction from the CPU 18 is transmitted. An ID inquiry is completed in this manner.

Then, on receiving the optical data inquiry from the CPU 18, the identification IC chip 201 transmits the ID data as return data. That is, the transmission data from the identification IC chip 201 is transmitted to the transmission and reception circuit 52 via the reverse route. This transmission data is demodulated by the transmission and reception circuit 52, and then transmitted to the CPU. Thus the series of communication related to the ID data reply is completed.

Optical data stored by the identification IC chip 201 is also fetched by the CPU 18 using a similar procedure. That is, firstly the CPU 18 generates a transmission signal for an optical data inquiry. Then the transmission and reception circuit 52 modulates it at a high frequency, and afterwards transmits it to the antenna 203 of the connecting section 31 via the antenna line 44.

On receiving the transmission signal, the antenna 203 transmits an electromagnetic signal to the identification IC chip 201. By this electromagnetic signal reaching the identification IC chip 201, an instruction from the CPU 18 is transmitted. An optical data inquiry is completed in this manner.

Then, on receiving an optical data inquiry from the CPU 18, the identification IC chip 201 transmits the optical data as return data. That is, the transmission data from the identification ID chip 201 is transmitted to the transmission and reception circuit 52 via the reverse route. This transmission data is demodulated by the transmission and reception circuit 52, and then transmitted to the CPU 18. Thus the series of communication related to optical data is completed.

Even in the case where there is data other than the ID and optical data to be read, it is fetched by the same procedure.

Here, the format of the communication data exchanged between the identification IC chip 201 and the CPU 18 is the same as in the first embodiment.

According to the endoscope device 1 of the present embodiment described above, it is possible to obtain the following effects.

The endoscope device 1 of the present embodiment uses a construction wherein its optical adaptor 32 incorporates an identification IC chip 201 in which optical data of the observation optical system 39 is stored, and an identification section 200 is provided on the control unit 6 side. Using this construction, it is possible to automate the operation of identifying the optical adaptor 32 without confirmation operations by the user. Accordingly, it is possible to identify the type of optical adaptor to be used accurately, thus enabling misoperation by the user to be prevented.

That is, in the endoscope device 1 of the present embodiment, since the optical characteristic values of the optical adaptor 32 to be used are held in the optical adaptor 32, it is not necessary to hold the optical data of the optical adaptor 32 on the control unit 6 side in advance. Accordingly, provided with only the identification IC chip 201, a calibration process for the registration and selection of optical data can be performed automatically with any optical adaptor. Once registered, from the next time it is possible to load corresponding environmental data into the RAM 20 by only detecting the ID. Thus it is possible to perform measurements immediately.

Furthermore, the endoscope device 1 of the present embodiment uses a construction wherein exchange of information between the identification IC chip 201 and the CPU 18 is performed without contact, using radio communication. Using this construction, electrical contact points are not required, which enables easy assembly. Furthermore, the non-contact system enables higher durability to be ensured than a contact system.

(Ninth Embodiment)

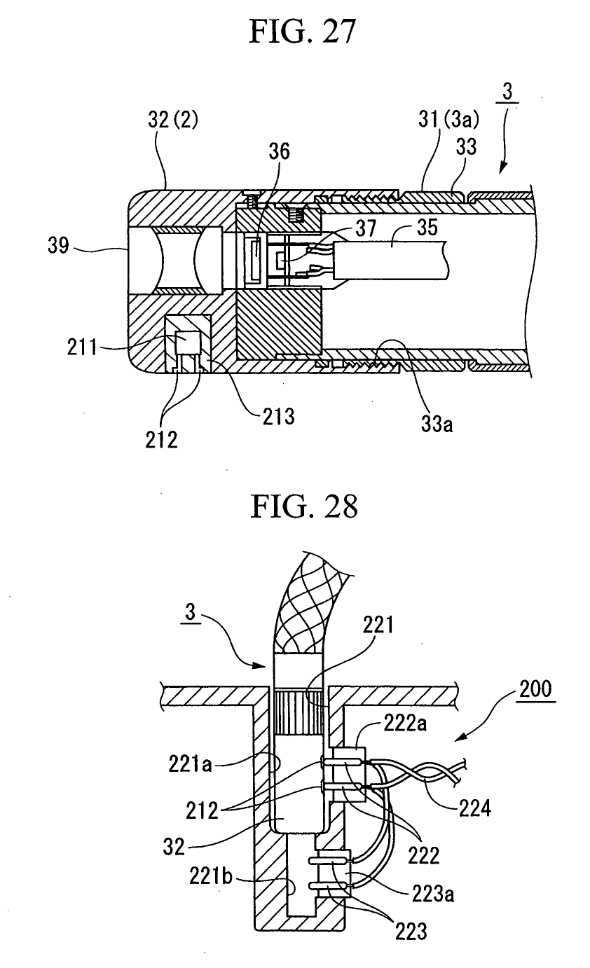

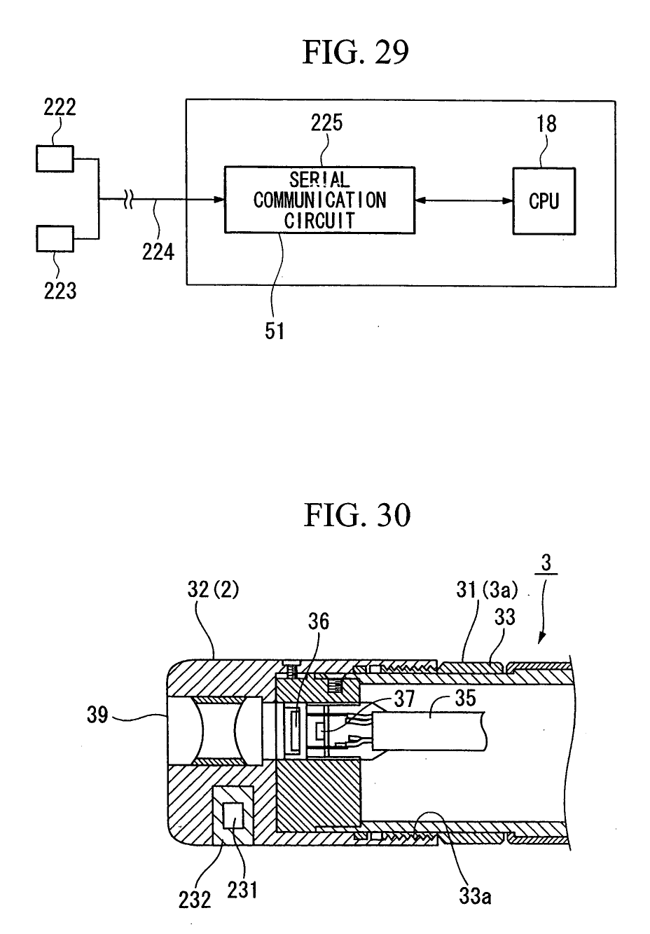

Hereunder is a description of a ninth embodiment of the present invention with reference to FIG. 27 to FIG. 29. FIG. 27 is a diagram showing the main parts of an endoscope device of the present embodiment, being a cross-sectional diagram showing the tip section of the endoscope insertion section, and an optical adaptor installed therein. Furthermore, FIG. 28 is a cross-sectional diagram showing a situation in which the optical adaptor of the endoscope device 1 is inserted in the identification section. Moreover, FIG. 29 is a block diagram of an electrical circuit provided in the endoscope device.

In the following description, the description focuses on the differences from the eighth embodiment, and the same symbols are used for the same structural elements as in the eighth embodiment, and their descriptions are omitted.

The distinguishing characteristic of the present embodiment is that information exchange between the identification IC chip 201 and the CPU 18 is performed by mechanical contact rather than the non-contact system in the eighth embodiment.

That is, as shown in FIG. 27, the identification IC chip (in the description hereunder, a new numeral 211 is assigned in order to distinguish it from the identification IC chip 201) is provided with a pair of IC side contact points 212 fixed in a support material 213 formed from epoxy resin. Furthermore, this identification IC chip 211 incorporates a CPU, which contains internal ROM and RAM. This identification IC chip 211 communicates with the outside using energy supplied from the communication line on the control unit 6, and also has a function of supplying optical information required for a calibration process to the outside.

At the other side, the identification section 200 of the present embodiment comprises a cavity 221 into which the optical adaptor 32 mounted on the tip section 3a is inserted, and first communication contacts 222 and second communication contacts 223 provided in this cavity 221 as shown in FIG. 28.

The cavity 221 is a hole provided in the surface of the panel of the control unit 6. The cavity 221 comprises a first insertion hole 221a into which an optical adaptor 32 with a relatively wide outside diameter is inserted, and a deeper second insertion hole 221b into which an optical adaptor 32 with a relatively narrow outside diameter is inserted.

The first insertion hole 221a contains the first pair of communication contacts 222, fixed in a contact supporting material 222a formed from epoxy resin. Therefore, in the case where an optical adaptor 32 mounted on a wide endoscope insertion section 3 is inserted, it makes contact with and conducts through the IC side contacts 212 of the identification IC chip 211. The first communication contacts 222 are connected to the CCU 17 via a two core communication line 224.

The second insertion hole 221b contains the second pair of communication contacts 223, fixed in a contact supporting material 223a formed from epoxy resin. Therefore, in the case where an optical adaptor 32 mounted on a narrow endoscope insertion section 3 is inserted, it makes contact with and conducts through the two IC side contacts 212 of the identification IC chip 211. The second communication contacts 223 are also connected to the CCU 17 via the two core communication line 224.

Furthermore, in the present embodiment, as shown in FIG. 29, a serial communication circuit 225 is used as the identification circuit 51 instead of the transmission and reception circuit 52. This communication circuit 225 transmits a communication signal from the CPU 18 to the first communication contacts 222 and the second communication contacts 223 via the two core communication line 224. Moreover, the communication signal is transmitted to the identification IC chip 211 via the IC side contacts 212 connected to either the first communication contacts 222 or the second communication contacts 223. Conversely, a communication signal from the identification IC chip 211 to the CPU 18 is transmitted via the reverse route.

In the endoscope device 1 of the present embodiment having the above-described construction, the IC side contacts 212 are connected to the first communication contacts 222 or the second communication contacts 223 mechanically by inserting the tip section 3a on which the optical adaptor 32 is mounted into the first insertion hole 221a or the second insertion hole 221b. Thus the connection is completed automatically. The flow of the calibration process performed after this is almost the same as the flow described in the first embodiment.

According to the endoscope device 1 of the present embodiment as described above, it is possible to obtain the same effect as in the eighth embodiment. That is, it is possible to automate the operation of identifying the optical adaptor 32 (stereo measurement optical adaptor 2) without confirmation operations by the user. Accordingly, it is possible to identify the type of optical adaptor 32 to be used accurately, thus enabling misoperation by the user to be prevented.

Furthermore, the endoscope device 1 of the present embodiment uses a construction in which communication data from the identification IC chip 211 to the CPU 18 is read via the connection between the IC side contacts 212, and the first communication contacts 222 or the second communication contacts 223. Using this construction, since communication data is read via mechanical contacts, it is possible to use a relatively large IC chip for the identification IC chip 211 compared with the case of wireless. In this manner, it is possible to increase the amount of data held on the optical adaptor 32 side.

(Tenth Embodiment)

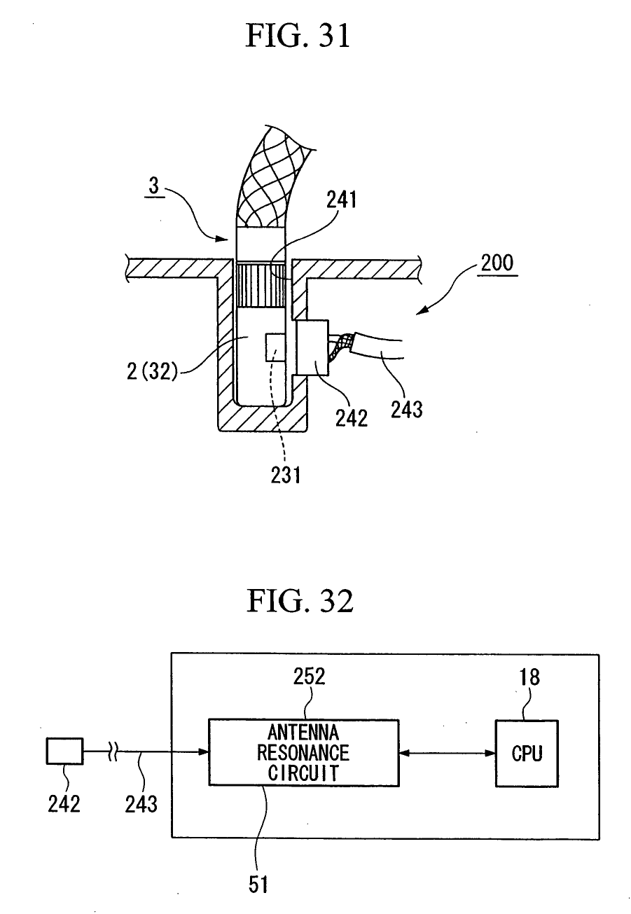

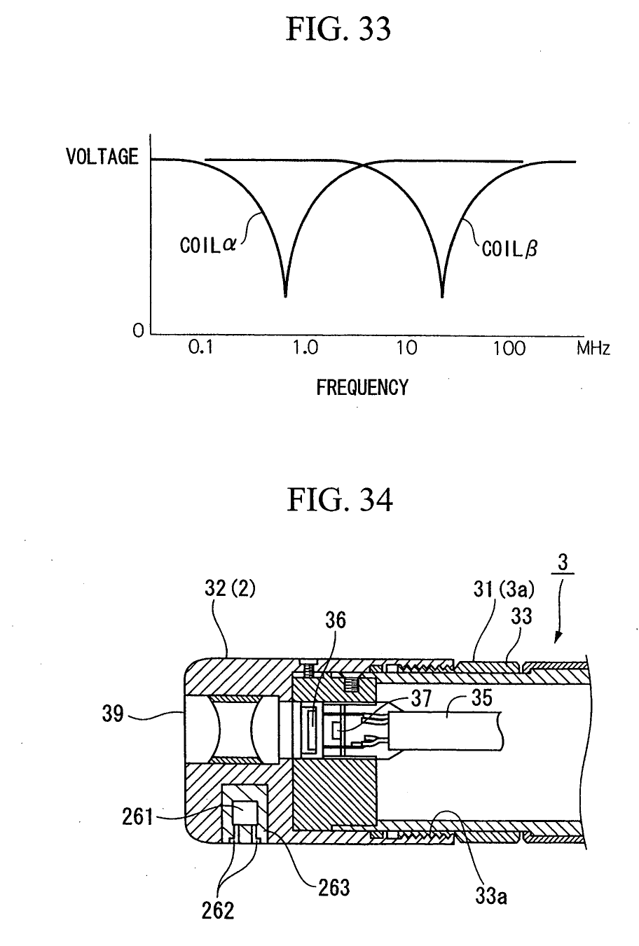

Hereunder is a description of a tenth embodiment of the present invention with reference to FIG. 30 to FIG. 33. FIG. 30 is a diagram showing the main parts of an endoscope device 1 of the present embodiment, being a cross-sectional diagram showing the tip section 3a of the endoscope insertion section 3, and an optical adaptor 32 (stereo measurement optical adaptor 2) installed therein. FIG. 31 is a cross-sectional diagram showing a situation in which the endoscope insertion section 3 on which the optical adaptor 32 is mounted is inserted in the identification section 200. FIG. 32 is a block diagram of an electrical circuit provided in the endoscope device 1. FIG. 33 is a graph showing the voltage of the electrical circuit of the endoscope device 1, wherein the frequency is on the horizontal axis, and the voltage in on the vertical axis.

In the following description, the description focuses on the differences from the eighth embodiment, and the same symbols are used for the same structural elements as in the eighth embodiment, and their descriptions are omitted.

The distinguishing characteristic of the present embodiment is that a combination of high frequency coils is used, instead of the combination of the identification IC chip 201 and the antenna 203 in the eighth embodiment, and optical adaptors 32 (stereo measurement optical adaptor 2) installed are identified by different resonance frequencies generated when they resonate.

That is, as shown in FIG. 30, a coil 231 fixed in a support material 232 formed from epoxy resin is incorporated in the optical adaptor 32, instead of the identification IC chip 201.

At the other side, the identification section 200 of the present embodiment comprises a cavity 241 into which the optical adaptor 32 mounted on the tip section 3a is inserted, and an antenna coil 242 provided in this cavity 241 as shown in FIG. 31.

The cavity 241 is a hole provided in the surface of the panel of the control unit 6. The antenna coil 242 is provided in a location in this cavity 241 aligned with the coil 231 when the optical adaptor 32 is inserted. This antenna coil 242 is connected to the CCU 17 via an antenna line 243 as shown in the figure.

Furthermore, in the present embodiment, as shown in FIG. 32, an antenna resonance circuit 252 is used as the identification circuit 51 instead of the transmission and reception circuit 52. This antenna resonance circuit 252 excites the antenna coil 242 at a predetermined frequency when it receives an instruction from the CPU 18. At the same time, the antenna resonance circuit 252 monitors the voltage at this time, and sends the voltage back to the CPU 18.

As shown in FIG. 33, in the case where a coil a with a high inductance is used as the coil 231, the resonance frequency is low. Conversely, in the case where a coil β with a low inductance is used as the coil 231, the resonance frequency is high. Accordingly, by checking the voltage as it increases and decreases depending on the height of the resonance frequency, it is possible to distinguish the type of optical adaptor 32 connected. The identification operation is performed using the same operations as in the so-called dipmeter principle.

To explain the operation of identifying this optical adaptor 32 with a specific example, firstly, the CPU 18 sends an instruction for an antenna resonance circuit 252 to excite the antenna coil 242 at 0.1 MHz, for example. Then, the antenna resonance circuit 252 excites the antenna coil 242 at 0.1 MHz, and also sends the voltage generated at that time back to the CPU 18. The CPU 18 stores the voltage, and sends an instruction to excite at 0.2 MHz this time. Changing the excitation frequency in this manner to 0.33 MHz, 0.35 MHz, 0.7 MHz, 1 MHz up to 700 MHz sequentially, the voltages are stored at each of the frequencies.

Subsequently, the CPU 18 searches the results for the frequency at which the voltage is highest, to determine the resonance frequency. The resonance frequency obtained in this manner can act as an identifier for identifying the optical adaptor 32 installed. Accordingly, by providing the type of the optical adaptor 32 corresponding to the resonance frequency, and its optical data, on the control unit 6 side in advance (providing it in the external storage medium), it is possible to select the optical data required for the calibration process. The calibration process performed after this is almost the same as the flow described in the first embodiment.

According to the endoscope device 1 of the present embodiment as described above, it is possible to obtain the same effect as in the eighth embodiment. That is, it is possible to automate the operation of identifying the optical adaptor 32 (stereo measurement optical adaptor 2) without confirmation operations by the user. Accordingly, it is possible to identify the type of optical adaptor 32 used accurately, thus enabling misoperation by the user to be prevented.

Furthermore, the endoscope device 1 of the present embodiment uses a construction in which the operation of identifying the optical adaptor 32 is performed by determining the resonance frequency generated between the coil 231 and the antenna coil 242. Using this construction, it is not necessary to use electrical contact points, it being only necessary to provide the coil 81 in the optical adaptor 32. Thus it is possible to assemble it easily. Moreover, it is possible to obtain information by a non-contact system, which also enables higher durability to be ensured than a contact system.

(Eleventh Embodiment)

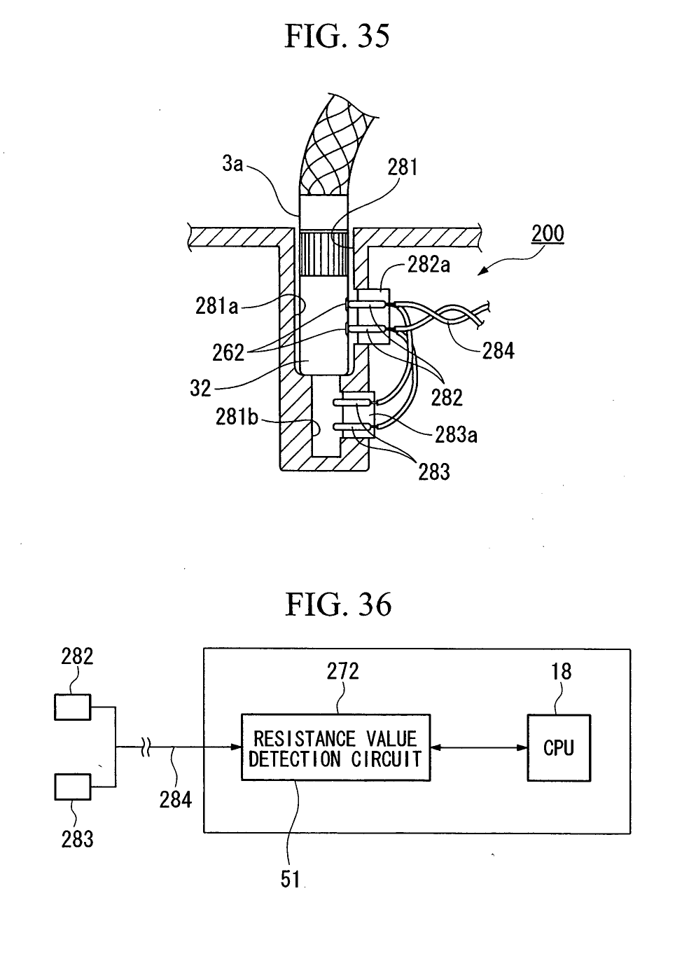

Hereunder is a description of an eleventh embodiment of the present invention with reference to FIG. 34 to FIG. 36. FIG. 34 is a diagram showing the main parts of an endoscope device 1 of the present embodiment, being a cross-sectional diagram showing the tip section 3a of the endoscope insertion section 3, and an optical adaptor 32 installed therein. FIG. 35 is a cross-sectional diagram showing a situation in which the endoscope insertion section 3 on which the optical adaptor 32 is mounted is inserted in the identification section 200. FIG. 36 is a block diagram of an electrical circuit provided in the endoscope device 1.

In the following description, the description focuses on the differences from the eighth embodiment, and the same symbols are used for the same structural elements as in the eighth embodiment, and their descriptions are omitted.

The distinguishing characteristic of the present embodiment is that a resistor is used instead of the identification IC chip 41 in the eighth embodiment, and the type of optical adaptor 32 is identified by obtaining the value of its resistance.

That is, as shown in FIG. 34, an identification resistor 261 is incorporated in the optical adaptor 32 instead of the identification IC chip 41. This identification resistor 261 has a pair of resistor side contacts 262 fixed in a support material 263 formed from epoxy resin.

At the other side, the identification section 200 of the present embodiment comprises a cavity 281 into which the optical adaptor 32 mounted on the tip section 3a is inserted, and first communication contacts 282 and second communication contacts 283 provided in this cavity 281 as shown in FIG. 35.

The cavity 281 is a hole provided in the surface of the panel of the control unit 6. The cavity 281 comprises a first insertion hole 281a into which an optical adaptor 32 with a relatively wide outside diameter is inserted, and a de eper second insertion hole 281b into which an optical adaptor 32 with a relatively narrow outside diameter is inserted.

The first insertion hole 281a contains the first pair of communication contacts 282, fixed in a contact supporting material 282a formed from epoxy resin. Therefore, in the case where an optical adaptor 32 mounted on a wide endoscope insertion section 3 is inserted, it makes contact with and conducts through the resistor side contacts 262 of the identification resistor 261. The first communication contacts 282 are connected to the CCU 17 via a two core communication line 284.

The second insertion hole 281b contains the second pair of communication contacts 283, fixed in a contact supporting material 283a formed from epoxy resin. Therefore, in the case where an optical adaptor 32 mounted on a narrow endoscope insertion section 3 is inserted, it makes contact with and conducts through the resistor side contacts 262 of the identification resistor 261. The second communication contacts 283 are also connected to the CCU 17 via the two core communication line 284.

Furthermore, in the present embodiment, as shown in FIG. 36, a resistance value detection circuit 252 is used as the identification circuit 51 instead of the transmission and reception circuit 52. This resistance value detection circuit 252 supplies a predetermined (constant) current to the identification resistor 261 via the communication line 284, and also has a function of sending the value of the voltage generated at that time to the CPU 18.

The voltage obtained in this manner can act as an identifier for identifying the optical adaptor 32 installed. Accordingly, by providing the type of the optical adaptor 32 corresponding to the voltage value, and its optical data, on the control unit 6 side in advance (providing it in the external storage medium), it is possible to select the optical data required for the calibration process. The calibration process performed after this is almost the same as the flow described in the eighth embodiment.

According to the endoscope device 1 of the present embodiment as described above, it is possible to obtain the same effect as in the eighth embodiment. That is, it is possible to automate the operation of identifying the optical adaptor 32 (stereo measurement optical adaptor 2) without confirmation operations by the user. Accordingly, it is possible to identify the type of optical adaptor 32 to be used accurately, thus enabling misoperation by the user to be prevented.

Moreover, the endoscope device 1 of the present embodiment uses a construction in which the operation of identifying the optical adaptor 32 is performed by reading a voltage value determined by the resistance value of the identification resistor 261. Using this construction, it is possible to set the value of resistance of the identification resistor 261 finely. Thus the construction may be such that the optical adaptors 32 can be identified easily even if there is a large number of types of them.

(Twelfth Embodiment)

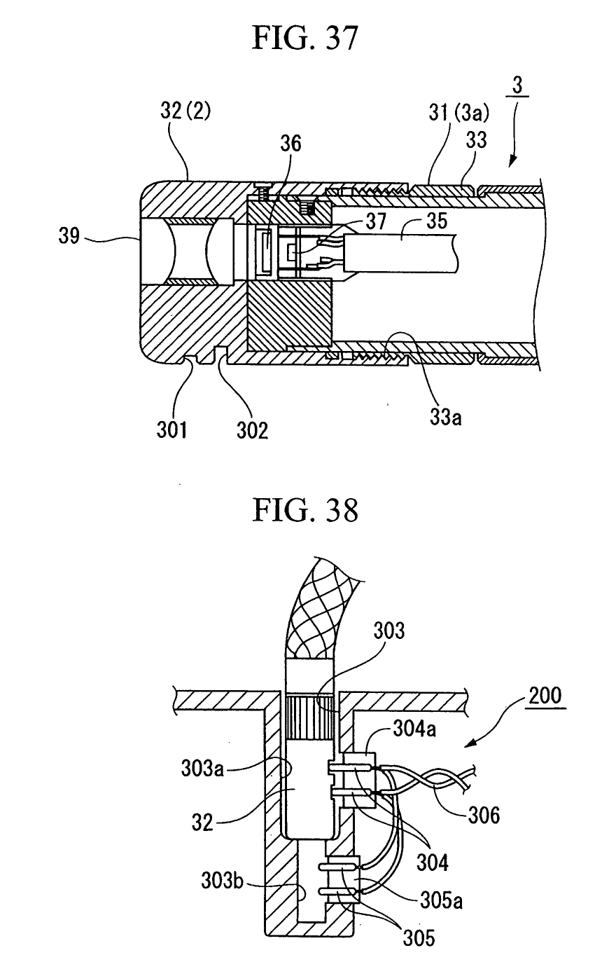

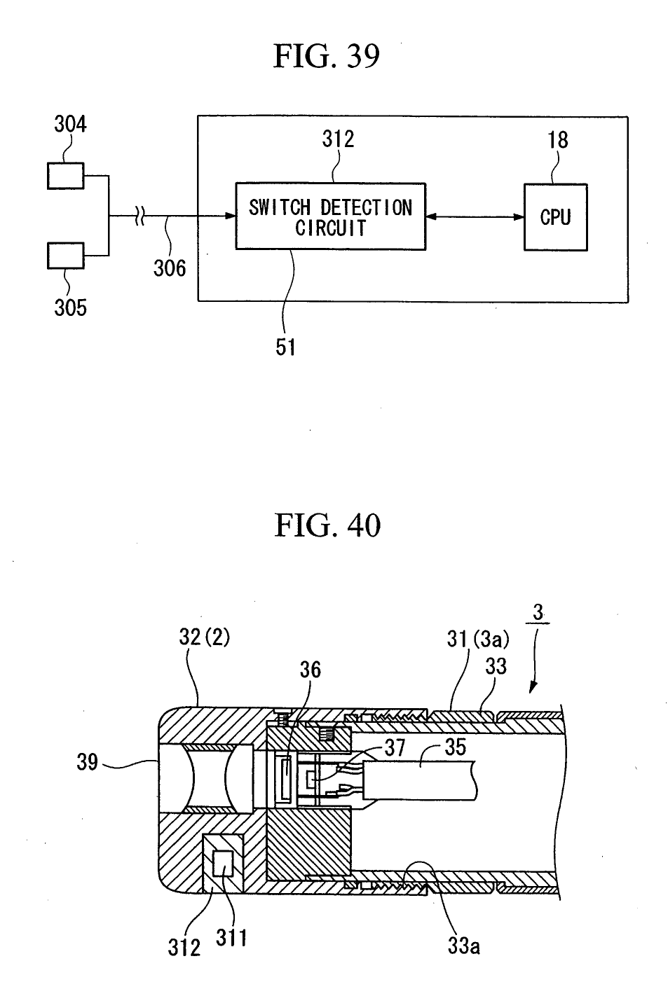

Hereunder is a description of a twelfth embodiment of the present invention with reference to FIG. 37 to FIG. 39. FIG. 37 is a diagram showing the main parts of an endoscope device 1 of the present embodiment, being a cross-sectional diagram showing the tip section 3a of the endoscope insertion section 3, and an optical adaptor 32 installed therein. FIG. 38 is a cross-sectional diagram showing a situation in which the endoscope insertion section 3 on which the optical adaptor 32 is mounted is inserted in the identification section 200. FIG. 39 is a block diagram of an electrical circuit provided in the endoscope device 1.

In the following description, the description focuses on the differences from the eighth embodiment, and the same symbols are used for the same structural elements as in the eighth embodiment, and their descriptions are omitted.

The distinguishing characteristic of the present embodiment is that mechanical switches are used for determining the optical adaptor 32, in contrast to the first embodiment.

That is, as shown in FIG. 37, a first identification cavity 301 and a second identification cavity 302 are formed in the optical adaptor 32 of the present embodiment facing toward the internal circumference of the identification section 200 into which it is inserted.

At the other side, the identification section 200 of the present embodiment comprises a cavity 303 into which the optical adaptor 32 mounted on the tip section 3a is inserted, and first identification switches (mechanical switches) 304 and second identification switches (mechanical switches) 305 provided in this cavity 303 as shown in FIG. 38.

The cavity 303 is a hole provided in the surface of the panel of the control unit 6. The cavity 303 comprises a first insertion hole 303a into which an optical adaptor 32 with a relatively wide outside diameter is inserted, and a deeper second insertion hole 303b into which an optical adaptor 32 with a relatively narrow outside diameter is inserted.

The first insertion hole 303a contains the first pair of identification switches 304, fixed in a switch supporting material 304a formed from epoxy resin. Therefore, in the case where an optical adaptor 32 mounted on a wide endoscope insertion section 3 is inserted, the first identification cavity 301 and the second identification cavity 302 make contact with it. The first identification switches 304 switch ON and OFF depending on the depths of the first identification cavity 301 and the second identification cavity 302 with which they make contact. Furthermore, the first identification switches 304 are connected to the CCU 17 via a signal line 306 as shown in the figure.

The second insertion hole 303b contains the second pair of identification switches 305, fixed in a switch supporting material 305a formed from epoxy resin. Therefore, in the case where an optical adaptor 32 mounted on a narrow endoscope insertion section 3 is inserted, the first identification cavity 301 and the second identification cavity 302 make contact with it. Similarly, the second identification switches 304 switch ON and OFF depending on the depths of the first identification cavity 301 and the second identification cavity 302 with which they make contact. Furthermore, the second identification switches 305 are connected to the CCU 17 via the two core signal line 306 as shown in the figure.

Moreover, in the present embodiment, as shown in FIG. 39, a switch detection circuit 312 is used as the identification circuit 51 instead of the transmission and reception circuit 52. This switch detection circuit 312 has a function of transmitting ON/OFF signals from the first identification switches 304 and the second identification switches 305 to the CPU 18.

Since two of both the first identification switches 304 and second identification switches 305 are provided, it is possible to determine four states by the combination of ON/OFF signals. However, in practice, one of them is a state in which an optical adaptor 32 is not installed, so it is eliminated. Thus it is possible to identify three types of optical adaptor 32.

Accordingly, the combinations of ON/OFF signals obtained in this manner can act as an identifier for identifying the optical adaptor 32 installed. As a result, by providing the type of the optical adaptor 32 corresponding to the ON/OFF signals, and its optical data, on the control unit 6 side in advance (providing it in the external storage medium), it is possible to select the optical data required for the calibration process. The calibration process performed after this is almost the same as the flow described in the first embodiment.

According to the endoscope device 1 of the present embodiment as described above, it is possible to obtain the same effect as in the first embodiment. That is, it is possible to automate the operation of identifying the optical adaptor 32 (stereo measurement optical adaptor 2) without confirmation operations by the user. Accordingly, it is possible to identify the type of optical adaptor 32 used accurately, thus enabling misoperation by the user to be prevented.

Moreover, in the endoscope device 1 of the present embodiment, only the first identification cavity 301 and the second identification cavity 302 need to be provided on the optical adaptor 32 side, which enables it to be use d easily and with low cost.

(Thirteenth Embodiment)

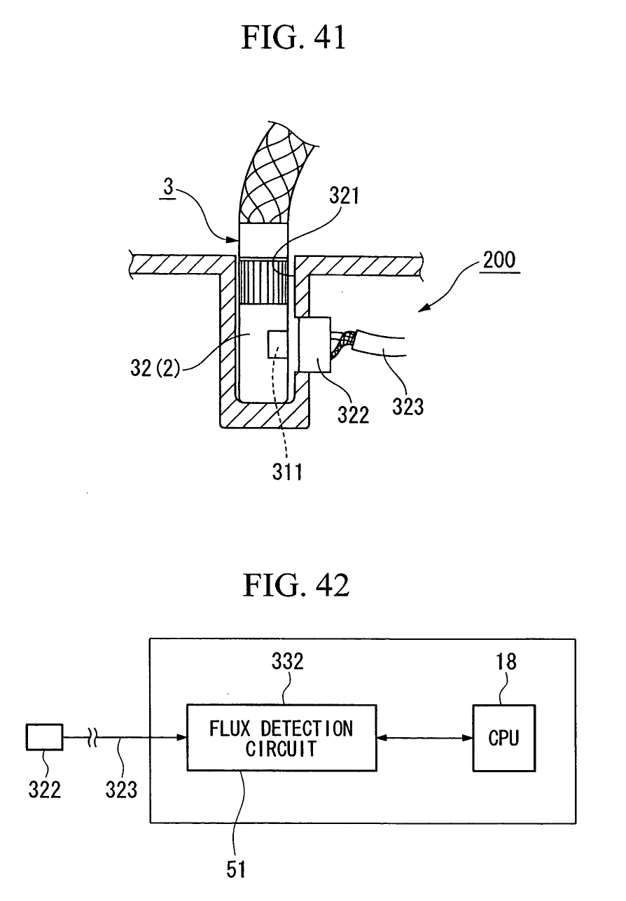

Hereunder is a description of a thirteenth embodiment of the present invention with reference to FIG. 40 to FIG. 42. FIG. 40 is a diagram showing the main parts of an endoscope device 1 of the present embodiment, being a cross-sectional diagram showing the tip section 3a of the endoscope insertion section 3, and an optical adaptor 32 installed therein. FIG. 41 is a cross-sectional diagram showing a situation in which the endoscope insertion section 3 on which the optical adaptor 32 is mounted is inserted in the identification section 200. FIG. 42 is a block diagram of an electrical circuit provided in the endoscope device 1.

In the following description, the description focuses on the differences from the eighth embodiment, and the same symbols are used for the same structural elements as in the eighth embodiment, and their descriptions are omitted.

The distinguishing characteristic of the present embodiment is that a combination of a magnet 311 and a hall element 322 is used, instead of the combination of the identification IC chip 41 and the antenna 43 in the eighth embodiment, and the type of optical adaptor 32 is identified by obtaining the strength and polarity of the magnet 311.

That is, as shown in FIG. 40, the optical adaptor 32 of the present embodiment is provided with the magnet 311 fixed in a support material 312 formed from an epoxy resin of a non-magnetic material.

At the other side, the identification section 200 of the present embodiment comprises a cavity 321 into which the optical adaptor 32 mounted on the tip section 3a is inserted, and the hall element 322 provided in this cavity 321 as shown in FIG. 41.

The cavity 321 is a hole provided in the surface of the panel of the control unit 6. The cavity 321 is provided with the hole element 322 in a location aligned with the magnet 311 when the optical adaptor 32 is inserted. This hole element 322 is connected to the CCU 17 via a connecting cable 323 as shown in the figure.

Moreover, in the present embodiment, as shown in FIG. 42, a flux detection circuit 252 is used as the identification circuit 51 instead of the transmission and reception circuit 52. This flux detection circuit 252 has a function of driving the hall element 322 and sending the flux level detected therein to the CPU 18. Accordingly, when the connecting section 31 on which the optical adaptor 32 is mounted is inserted, the flux density detected by the hall element 322 changes due to the magnetic field generated by the magnet 311. The flux density (strength and polarity of the magnet 311) obtained in this manner can act as an identifier for identifying the optical adaptor 32 installed. Accordingly, by providing the type of the optical adaptor 32 corresponding to the flux density, and its optical data, on the control unit 6 side in advance (providing it in the external storage medium), it is possible to select the optical data required for the calibration process. The calibration process performed after this is almost the same as the flow described in the first embodiment.

According to the endoscope device 1 of the present embodiment as described above, it is possible to obtain the same effect as in the eighth embodiment. That is, it is possible to automate the operation of identifying the optical adaptor 32 (stereo measurement optical adaptor 2) without confirmation operations by the user. Accordingly, it is possible to identify the type of optical adaptor 32 used accurately, thus enabling misoperation by the user to be prevented.

Furthermore, the endoscope device 1 of the present embodiment does not require electrical contact points. Thus it is possible to assemble it easily. Moreover, it is possible to obtain information by a non-contact system, which also enables higher durability to be ensured than a contact system.

(Fourteenth Embodiment)

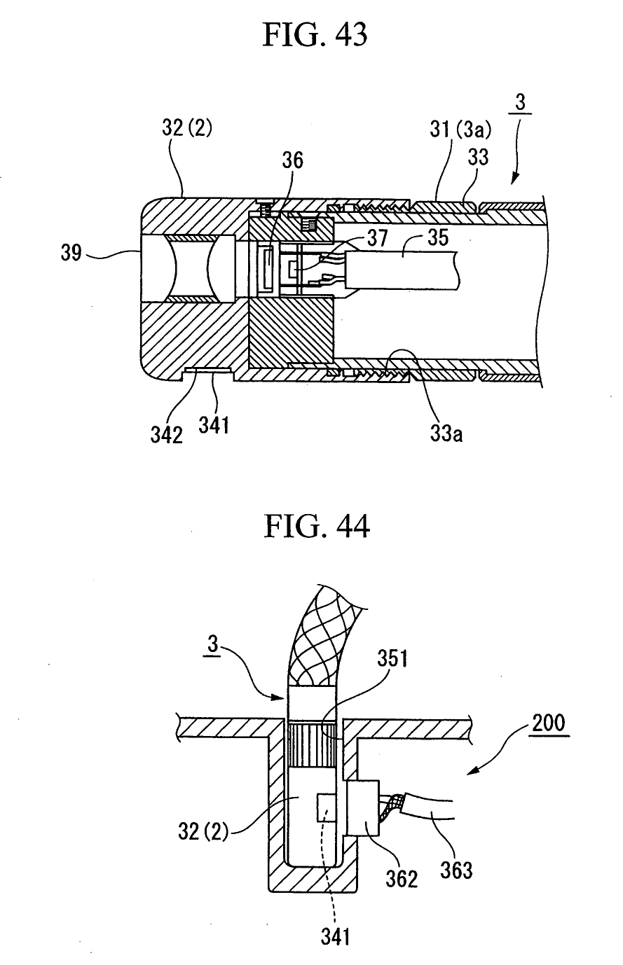

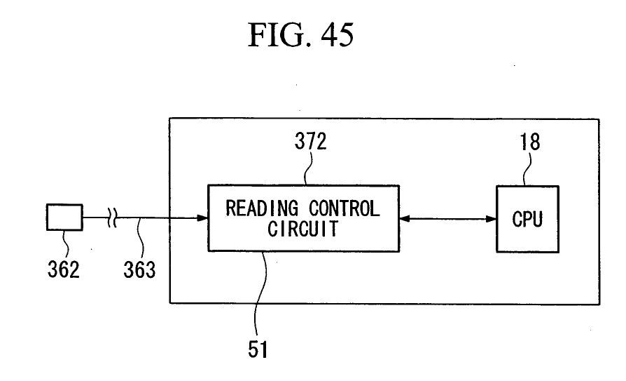

Hereunder is a description of a fourteenth embodiment of the present invention with reference to FIG. 43 to FIG. 45. FIG. 43 is a diagram showing the main parts of an endoscope device 1 of the present embodiment, being a cross-sectional diagram showing the tip section 3a of the endoscope insertion section 3, and an optical adaptor 32 installed therein. FIG. 44 is a cross-sectional diagram showing a situation in which the endoscope insertion section 3 on which the optical adaptor 32 is mounted is inserted in the identification section 200. FIG. 45 is a block diagram of an electrical circuit provided in the endoscope device 1.

In the following description, the description focuses on the differences from the eighth embodiment, and the same symbols are used for the same structural elements as in the eighth embodiment, and their descriptions are omitted.

The distinguishing characteristic of the present embodiment is that a combination of a character/image information display section 341 and a receiving device 362 is used, instead of the combination of the identification IC chip 41 and the antenna 43 in the eighth embodiment, and the type of optical adaptor 32 is identified based on character/image information.

That is, as shown in FIG. 43, the character/image information display section 341, in which character/image information is written on the side face of an elongated rod shaped or a flat material, is fixed on the side face 342 of the optical adaptor 32 of the present embodiment.

At the other side, the identification section 200 of the present embodiment comprises a cavity 351 into which the optical adaptor 32 mounted on the tip section 3a is inserted, and the receiving device 362 provided in this cavity 351 as shown in FIG. 44.

The cavity 351 is a hole provided in the surface of the panel of the control unit 6. The receiving device 362 is provided in a location in this cavity 351 aligned with the character/image display section 341 when the optical adaptor 32 is inserted. This receiving device 362 is connected to the CCU 17 via a signal line 363 as shown in the figure.

Furthermore, in the present embodiment, as shown in FIG. 45, a reading control circuit 372 is used as the identification circuit 51 instead of the transmission and reception circuit 52. This reading control circuit 372 has a function of communicating with the receiving device 362, and sending the character/image information thus detected to the CPU 18. Accordingly, when the connecting section 31 on which the optical adaptor 32 is mounted is inserted in the cavity 351, the character/image information display section 341 faces the receiving device 362. Thus the receiving device 362 reads its character/image information, and converts it to a digital signal. Then, this digital signal is transmitted to the CPU 18 via the signal line 363.

The character/image information obtained in this manner can act as an identifier for identifying the optical adaptor 32 installed. Accordingly, by providing the type of the optical adaptor 32 corresponding to the character/image information, and its optical data, on the control unit 6 side in advance (providing it in the external storage medium), it is possible to select the optical data required for the calibration process. The calibration process performed after this is almost the same as the flow described in the first embodiment.

According to the endoscope device 1 of the present embodiment as described above, it is possible to obtain the same effect as in the eighth embodiment. That is, it is possible to automate the operation of identifying the optical adaptor 32 (stereo measurement optical adaptor 2) without confirmation operations by the user. Accordingly, it is possible to identify the type of optical adaptor 32 used accurately, thus enabling misoperation by the user to be prevented.

Furthermore, the endoscope device 1 of the present embodiment does not require electrical contact points. Thus it is possible to assemble it easily. Moreover, it is possible to obtain information by a non-contact system, which also enables higher durability to be ensured than a contact system.

In the endoscopes of the first embodiment to the fourteenth embodiment, a CCD 36 is used as an imager on the tip of the endoscope insertion section 3. However, the invention is not limited to this, and a C-MOS image sensor may be used. Furthermore, a light receiving section may be constructed using a bundle of optical fibers.

Moreover, only the ID (identifier) of an optical adaptor 32 is read from the optical adaptor 32, and when inputting the optical data corresponding to this ID into the control unit 6, it is read from the external memory medium in the above-described embodiments. However, it is not limited to this external memory medium, and a hard disc drive may be provided in the control unit 6, on which optical data is provided in advance. Furthermore, optical data may be input into the control unit 6 via a communication line, such as from the Internet.

1. An optical adaptor that is detachably installed at the tip of an endoscope insertion section having a light receiving section at the tip thereof, said optical adapter comprising;
an optical system which forms an image in said light receiving section; and
an information device containing at least one of information for identifying itself and optical characteristic information.

2. An optical adaptor according to claim 1, wherein said information device is an identification IC chip.

3. An endoscope device comprising;
an endoscope insertion section having a light receiving section at the tip thereof;
an optical adaptor that is detachably installed at the tip of said endoscope insertion section, and having an optical system which forms an image in said light receiving section, and an information device containing at least one of information for identifying itself and optical characteristic information; and
a reading section which is installed in the tip of said endoscope insertion section and obtains said information.

4. An endoscope device according to claim 3, wherein reading of said information from said optical adaptor to said reading section is performed by wireless transmission.

5. An endoscope device according to claim 4, wherein
said optical adaptor comprises an identification IC chip,
said reading section comprises an antenna, and
reading of said information is performed by said wireless transmission between said identification IC chip, and said antenna.

6. An endoscope device according to claim 3, wherein
said optical adaptor comprises a joining terminal,
said reading section comprises a joining terminal, and
reading of said information from said optical adaptor to said reading section is performed via a connection between the joining terminal of said optical adaptor and the joining terminal of said reading section.

7. An endoscope device according to claim 6, wherein
said optical adaptor comprises an identification IC chip, and
reading of said information is performed via said connection between a joining terminal of said identification IC chip and the joining terminal of said reading section.

8. An endoscope device according to claim 3, wherein
said optical adaptor comprises a coil,
said reading section comprises a coil, and
reading of said information from said optical adaptor to said reading section is performed by reading a resonance frequency between the coil of said optical adaptor and the coil of said reading section.

9. An endoscope device according to claim 3, wherein
said optical adaptor comprises a resistor, and
reading of said information from said optical adaptor to said reading section is performed by reading electrical resistivity of said resistor.

10. An endoscope device according to claim 3, wherein
reading of said information from said optical adaptor to said reading section is performed by reading a concave or convex shape formed in said optical adaptor.

11. An endoscope device according to claim 3, wherein
said optical adaptor comprises a magnetic material, and
reading of said information from said optical adaptor to said reading section is performed by reading a flux level of said magnetic material.

12. An endoscope device according to claim 3, wherein
said optical adaptor comprises an information display section, and
reading of said information from said optical adaptor to said reading section is performed by reading information of said information display section.

13. An endoscope device comprising:
a main body;
an endoscope insertion section, which is connected to the main body and has a light receiving section at the tip thereof;
an optical adaptor that is detachably installed at the tip of said endoscope insertion section, and having an optical system which forms an image in the light receiving section, and an information device containing at least one of information for identifying itself and optical characteristic information; and
a reading section which is installed in said main body and obtains said information from said optical adaptor.

14. An endoscope device according to claim 13, wherein reading of said information from said optical adaptor to said reading section is performed by wireless transmission.

15. An endoscope device according to claim 14, wherein
said optical adaptor comprises an identification IC chip,
said reading section comprises an antenna, and
reading of said information is performed by said wireless transmission between said identification IC chip, and said antenna.

16. An endoscope device according to claim 13, wherein
said optical adaptor comprises a joining terminal,
said reading section comprises a joining terminal, and
reading of said information from said optical adaptor to said reading section is performed via a connection between the joining terminal of said optical adaptor and the joining terminal of said reading section.

17. An endoscope device according to claim 16, wherein
said optical adaptor comprises an identification IC chip, and
reading of said information is performed via said connection between a joining terminal of said identification IC chip and the joining terminal of said reading section.

18. An endoscope device according to claim 13, wherein
said optical adaptor comprises a coil,
said reading section comprises a coil, and
reading of said information from said optical adaptor to said reading section is performed by reading a resonance frequency between the coil of said optical adaptor and the coil of said reading section.

19. An endoscope device according to claim 13, wherein
said optical adaptor comprises a resistor, and
reading of said information from said optical adaptor to said reading section is performed by reading electrical resistivity of said resistor.

20. An endoscope device according to claim 13, wherein
reading of said information from said optical adaptor to said reading section is performed by reading a concave or convex shape formed in said optical adaptor.

21. An endoscope device according to claim 13, wherein
said optical adaptor comprises a magnetic material, and
reading of said information from said optical adaptor to said reading section is performed by reading a flux level of said magnetic material.

22. An endoscope device according to claim 13, wherein
said optical adaptor comprises an information display section, and
reading of said information from said optical adaptor to said reading section is performed by reading information of said information display section.
